(19) United States
(12) Patent Application Publication
(10) Pub. No.: US 2016/0145636 A1
(43) Pub. Date: May 26, 2016

(54) COMPOSITIONS AND METHODS FOR THE PRODUCTION OF GLUTEN FREE FOOD PRODUCTS

(71) Applicant: JM BIOLOGICALS, Somerset, NJ (US)

(72) Inventors: Joachim Messing, Somerset, NJ (US); Paul J. Ciclitira, London (GB)

(21) Appl. No.: 14/889,950

(22) PCT Filed: May 8, 2014
(86) PCT No.: PCT/US14/37296

§ 371 Date: Nov. 9, 2015

Related U.S. Application Data
(60) Provisional application No. 61/820,893, filed on May 8, 2013.

(51) Int. Cl. C12N 15/82; G01N 33/50; C07K 14/415

(52) U.S. Cl. CPC C12N 15/8251; C07K 14/415; C12N 15/8257; G01N 33/505; G01N 33/5026; C12N 15/8218; A23L 1/1041; A23V 2002/00

(57) ABSTRACT

Compositions and methods for the production of baked goods and flour, which do not induce CD are disclosed.

This application claims priority to U.S. Provisional Application No. 61/820,893 filed May 8, 2013, the entire contents being incorporated by reference as though set forth in full.

### FIELD OF THE INVENTION

This invention is related to the fields of transgenic plants and the production of gluten-free food products for human consumption. More specifically, the invention provides transgenic plants expressing recombinant glutenins and gliadins and the means to produce flour that does not induce undesirable immune reactions upon ingestion for the prevention and management of celiac disease (CD).

### BACKGROUND OF THE INVENTION

Several publications and patent documents are cited throughout the specification in order to describe the state of the art to which this invention pertains. Each of these citations is incorporated by reference herein as though set forth in full.

Gluten, the major protein component of wheat can be divided into the monomeric, alcohol soluble gliadins and the polymeric alcohol insoluble glutenins, the latter comprising the LMW-GS and HMW-GS. HMW-GS cause CD in vivo (Dewar et al., 2006). LMW-GS stimulate gluten sensitive T-cells in vitro (Vader et al., 2002) and are assumed to be CD-toxic, although no in vivo studies exist. The generally accepted pathogenesis of CD involves an abnormal immune response, both adaptive and innate, to gluten protein antigens, some of which have been modified by small intestinal tissue transglutaminase (tTG). The latter causes selective partial deamidation of gluten proteins with glutamine (Q) being transformed into glutamic acid (E) resulting in pathology inducing neo-epitopes. The relationship between CD and gluten protein structure has been investigated with in vitro and in vivo studies in patient volunteers. Most toxic gliadin peptides are derived from the repetitive domain of gliadin proteins, contained in the N-terminal sequence, that are mainly comprised of glutamine, proline and aromatic amino acids (Arentz-Hansen et al., 2000; Arentz-Hansen 2002). Two celiac toxic epitopes have been identified in HMW-GS, using gluten sensitive T-cell lymphocyte transformation assays (Van de Wal et al. 1999; Vader et al. 2002). Recent evidence using peripheral blood lymphocytes from gluten challenged celiac patients suggests that the glutenins are non-immunostimulatory to celiac gluten sensitive T-cell lymphocytes (Tye-Din et al. 2010). Mitea (Mitea et al. 2010) has described a universal approach to eliminate antigenic properties of alpha gliadin peptides in CD but which does not address the celiac toxicity of the glutenin protein. To date a total of 31 toxic epitopes have been identified (Sollid et al. 2012).

Gluten proteins, both gliadins and glutenins are essential contributors to the rheological properties of dough, although their functions are different. Gliadins contribute mainly to the viscosity and extensibility of dough, whereas glutenins are responsible for dough strength and elasticity. The gliadins comprise three sub-fractions, termed α, γ and ω with similar functional properties. The glutenin fraction comprises high-molecular-weight subunits (HMW-GS) and low-molecular-weight subunits (LMW-GS). Both HMW-GS and LMW-GS are linked by intermolecular disulphide bonds enabling production of polymers that reach molecular weights of up to several million. The structure of HMW-GS influences dough properties and wheat quality significantly more than LMW-GS. The combination of HMW-GS 1Dx5 and 1Dy10 produces the best bread making qualities (Payne et al, 1984).

When people with CD eat foods containing gluten, their immune system responds by damaging the small intestine. Specifically, tiny fingerlike protrusions, called villi, on the lining of the small intestine are lost. Normally, nutrients from food are absorbed into the bloodstream through these villi. Without villi, a person becomes malnourished—regardless of the quantity of the food eaten. Symptoms of CD may include one or more of the following: chronic diarrhea, weight loss, pale foul-smelling stool, unexplained anemia, recurring abdominal bloating and pain, bone pain, behavior changes, muscle cramps, fatigue, delayed growth, failure to thrive in infants, pain in the joints, seizures, tingling numbness in the legs resulting from nerve damage, pale sores inside the mouth known as aphthus ulcers, skin rash known as dermatitis, herpetiformis, tooth discoloration or loss of enamel, and missed menstrual periods.

It is an object of the invention to provide consumable maize-based baking flour that is incapable of inducing the undesired immune responses described above.

### SUMMARY OF THE INVENTION

In accordance with the present invention, a method for the production of transgenic maize, which expresses wheat glutenin and gliadin proteins lacking CD inducing epitopes, is provided. In a first aspect, CD-inducing epitopes present in wheat glutenin and gliadin are identified in bioassays using gluten sensitive T cells. Recombinant nucleic acids are then synthesized encoding functional wheat glutenin and gliadin proteins, which lack epitopes associated with the occurrence of CD. Transgenic maize plants expressing these nucleic acids are then produced.

An exemplary method entails introducing a DNA construct comprising sequences encoding one or more wheat glutenin or gliadin proteins into maize cells wherein the sequences have been genetically altered such that the encoded proteins lack native CD-inducing epitopes. The construct could optionally comprise a selectable marker suitable for isolation of transgenic cells. After transformation the isolated cells are propagated to generate a transgenic maize plant. Flour obtained from the plants can then be used for baking improved consumable products, said products lacking CD inducing epitopes and thereby being safe to consume by patients exhibiting gluten intolerance.

In an alternative embodiment, the method can further comprise back crossing the resulting first transgenic plant with a separate, second transgenic plant expressing at least one different recombinant glutenin or gliadin protein, thereby producing a plant expressing altered glutenins and gliadins from said first and second plants. In another embodiment the method can comprise introduction of at least one RNAi construct into said plant, said RNAi molecule being effective to down modulate production of at least one zein protein. Finally, in a preferred embodiment, the transgenic maize is obtained from a high quality protein maize line, thereby providing maize exhibiting improved nutritional properties. Flour obtained from the transgenic maize is also encompassed by the present invention as are plants or progeny obtained from the methods described above.

### BRIEF DESCRIPTION OF THE DRAWINGS

FIG. 1 is a schematic diagram of the chimeric CD epitope free transgenic glutenin and gliadin encoding nucleic acids of the invention.

FIG. 2 provides the sequence information for the native immunogenic alpha gliadin sequence and an exemplary altered (syn) sequence of the invention. Known toxic motifs are highlighted in bold.

FIG. 3 provides the sequence information for the native immunogenic gamma gliadin sequence and an exemplary altered (syn) sequence of the invention. Known toxic motifs are highlighted in bold.

FIG. 4 provides the sequence information for the native immunogenic HMW glutenin sequence and an exemplary altered (syn) sequence of the invention. Known toxic motifs are highlighted in bold.

FIG. 5 provides the sequence information for the native immunogenic LMW glutenin sequence and an exemplary altered (syn) sequence of the invention. Known toxic motifs are highlighted in bold.

FIG. 6 provides the sequence information for omega gliadin and omega gamma gliadin sequences of the invention. Known toxic motifs are highlighted in bold.

FIG. 7 provides the vector map of an expression vector suitable for transduction of targeted plant cells for the creation of transgenic plants expressing the transgenic proteins of the invention. The vector pTF102 is used in Agrobacterium-mediated transformation of maize. See B. H. Frame, B., H. Shou, R. K., Chikwamba, Z. Zhang, C. Xiang et al., 2002 Agrobacterium tumefaciens-mediated transformation of maize embryos using a standard binary vector system. Plant Physiol 129: 13-22.

FIG. 8 provides the vector map for expression of the inhibitory RNAi targeting maize zein proteins. Construction of various RNAi constructs against gamma and alpha zein genes has been published previously Wu, Y., et al.,(2010) Gamma-Zeins are essential for endosperm modification in quality protein maize. Proc Natl Acad Sci U S A 107: 12810-12815.

FIG. 9 is a schematic diagram showing the genetic crosses suitable for production of maize expressing recombinant gliadins and glutenins from wheat, which do not induce unwanted immune responses. Transgenic events can be produced as single or multiple events. Single events can then be combined by conventional crosses. The synthetic glutenin and gliadin are combined to test baking quality. Synthetic prolamins (glutenin or gliadin) are then combined with gamma RNAi because gamma zeins are the closest to glutenin and gliadin and could substitute their role in maize endosperm. (Xu, J.-H., and Messing, J. (2009). Amplification of prolamin storage protein genes in different subfamilies of the Poaceae. Theor Appl Genet 119, 1397-1412). Reduction of alpha zeins with RNAi could increase the level of wheat prolamins. Because alpha RNAi gives rise to non-vitreous maize kernels, wheat prolamins could optionally be used to select for quality protein maize (QPM). Wu et al., (supra). This approach should result in the production of maize that exhibits enhanced baking properties in conjunction with improved nutritional qualities.

### DETAILED DESCRIPTION OF THE INVENTION

CD is an inflammatory disease of the small intestine and is triggered by dietary components that are present in the storage proteins (gluten) of wheat, rye, barley and possibly oats. It is estimated that CD affects approximately 1% of individuals in Europe and the US. Treatment involves a strict, lifelong gluten-free diet with withdrawal of these cereals. Elimination of wheat-based products causes severe restriction in quality of life. Surrogates for wheat include maize flour that exhibits poor sensory characteristics, high cost and poor dietary compliance.

We describe herein the means to identify the toxic epitopes in wheat glutenin and gliadin proteins which give rise to the aberrant immune response observed in CD. We also describe recombinant techniques for the production of transgenes encoding altered glutenin and gliadin proteins, which lack such epitopes. These transgenes will be used to produce recombinant maize, which express modified wheat glutens that do not induce CD. Flour produced from the transgenic maize of the invention will then be utilized for the production of baked goods, which can be safely consumed by the celiac patient and other patients who exhibit reduced tolerance to gluten consumption.

Thus, in accordance with the present invention, a genetically modified maize that cannot only provide flour with the baking and sensorial qualities of wheat, but which also does not exacerbate CD that affects millions of people in Europe and North America is disclosed. This approach should also be effective to improve the palatability of maize based gluten-free bread.

### DEFINITIONS

The term “celiac disease” encompasses a spectrum of conditions caused by varying degrees of gluten sensitivity, including a severe form characterized by a flat small intestinal mucosa (hyperplastic villous atrophy) and other forms characterized by milder symptoms.

As used herein, “genetically modified” or “genetically altered” means the modified expression of a gluten protein resulting from one or more genetic modifications; the modifications including but not limited to: recombinant gene technologies, induced mutations, and breeding stably genetically modified plants to produce progeny and seed comprising the altered gene product.

Transgenic plants producing seeds and grain with altered gluten protein content are also provided.

The term “decreased” is intended to mean that the measurement of a parameter is changed by at least 10%, 20%, 30%, 40%, 50%, 60%, 70%, 80%, 90%, 100%, 200% or more when compared to the measurement of that parameter in a suitable control.

The terms “inhibit,” “inhibition,” “inhibiting”, “reduced”, “reduction” and the like as used herein refer to any decrease in the expression or function of a target gene product, including any relative decrement in expression or function up to and including complete abrogation of expression or function of the target gene product.

The term “expression” as used herein in the context of a gene product refers to the biosynthesis of that gene product, including the transcription and/or translation of the gene product. Inhibition of expression or function of a target gene product (i.e., a gene product of interest) can be in the context of a comparison between any two plants, for example, expression or function of a target gene product in a genetically altered plant versus the expression or function of that target gene product in a corresponding wild-type plant. Alternatively, inhibition of expression or function of the target gene product can be in the context of a comparison between plant cells, organelles, organs, tissues, or plant parts within the same plant or between plants, and includes comparisons between developmental or temporal stages within the same plant or between plants. Any method or composition that down-regulates expression of a target gene product, either at the level of transcription or translation, or down-regulates functional activity of the target gene product can be used to achieve inhibition of expression or function of the target gene product.

The term “inhibitory sequence” encompasses any polynucleotide or polypeptide sequence that is capable of inhibiting the expression of a target gene product, for example, at the level of transcription or translation, or which is capable of inhibiting the function of a target gene product. Exemplary constructs encoding such inhibitory sequences are disclosed herein.

When the phrase “capable of inhibiting” is used in the context of a polynucleotide inhibitory sequence, it is intended to mean that the inhibitory sequence itself exerts the inhibitory effect; or, where the inhibitory sequence encodes an inhibitory nucleotide molecule (for example, hairpin RNA, miRNA, or double-stranded RNA polynucleotides), or encodes an inhibitory polypeptide (i.e., a polypeptide that inhibits expression or function of the target gene product), following its transcription (for example, in the case of an inhibitory sequence encoding a hairpin RNA, miRNA, or double-stranded RNA polynucleotide) or its transcription and translation (in the case of an inhibitory sequence encoding an inhibitory polypeptide), the transcribed or translated product, respectively, exerts the inhibitory effect on the target gene product (i.e., inhibits expression or function of the target gene product).

Conversely, the terms “increase”, “increased”, and “increasing” in the context of the methods of the present invention refer to any increase in the expression or function of a gene product, including any relative increment in expression or function.

In many instances the nucleotide sequences for use in the methods of the present invention, are provided in transcriptional units with for transcription in the plant of interest. A transcriptional unit is comprised generally of a promoter and a nucleotide sequence operably linked in the 3′ direction of the promoter, optionally with a terminator.

“Operably linked” refers to the functional linkage between a promoter and a second sequence, wherein the promoter sequence initiates and mediates transcription of the DNA sequence corresponding to the second sequence. The expression cassette will include 5′ and 3′ regulatory sequences operably linked to at least one of the sequences of the invention.

Generally, in the context of an over expression cassette, operably linked means that the nucleotide sequences being linked are contiguous and, where necessary to join two or more protein coding regions, contiguous and in the same reading frame. In the case where an expression cassette contains two or more protein coding regions joined in a contiguous manner in the same reading frame, the encoded polypeptide is herein defined as a “heterologous polypeptide” or a “chimeric polypeptide” or a “fusion polypeptide”. The cassette may additionally contain at least one additional coding sequence to be co-transformed into the organism. Alternatively, the additional coding sequence(s) can be provided on multiple expression cassettes.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process, which includes the construction of a cDNA library from mRNA and isolation of distinct cDNA clones, yields an approximately 10⁻⁶-fold purification of the native message. Thus, purification of at least one order of magnitude, preferably two or three orders, and more preferably four or five orders of magnitude is expressly contemplated. Thus the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the recombinant nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the gluten-specific coding nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is readily measurable, e.g., by biological assay, immunoassay, radio immunoassay, or by colorimetric, fluorogenic, chemiluminescent or other methods. GFP is exemplified herein. The nucleic acid may be either RNA or DNA, linear or circular, single or double stranded, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional-termination signals and the like.

The term “selectable marker gene” refers to a gene that when expressed confers a selectable phenotype, such as herbicide tolerance, on a transformed plant cell.

The terms “recombinant plant,” or “transgenic plant” refer to plants, which have a new combination of genes or nucleic acid molecules. A new combination of genes or nucleic acid molecules can be introduced into a plant using a wide array of nucleic acid manipulation techniques available to those skilled in the art.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins, with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp), which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample, which may be tested for a particular molecule or cellular response, preferably the sample comprises T cells, which can be tested for undesirable immune responses. Samples may include but are not limited to cells, body fluids, including blood, serum, plasma, urine, saliva, tears, pleural fluid and the like.

The term “rheology” refers to empirical rheological measurements including farinograms and extensograms. The results collected will allow determining the influence of the grain composition on water adsorption, mixing profiles, stability and extensibility of the doughs. These empirical data will be compared to fundamental rheological values obtained from dynamic oscillatory mode of measurement (determination of complex viscosity, complex modulus, and phase angle).

The “ultra structure” of the grains as well as the dough and cereal products will be assessed by using Scanning electron microscopy and laser and scanning microscopy. The interaction individual dough components can be monitored by using specific dyes which selectively visualise protein, carbohydrates, etc. The three dimensional structure will be visualised with specific image analysis software.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule, which exhibits the capacity to modulate the activity or immunogenicity of the altered gluten encoding nucleic acids described herein or their encoded proteins. Agents are evaluated for potential biological activity by inclusion in screening assays described hereinbelow.

The following materials and methods are provided to facilitate the practice of the present invention. They are not intended to limit the invention in any way.

### Cell Based Assay for Identification of CD-Associated Epitopes in Wheat Glutens

In vitro duodenal biopsy culture: Test peptides, peptic/tryptic gluten and ovalbumin, the latter two serving as positive and negative controls will be dissolved at 200 μg/ml for single peptides and 1 mg/ml for peptide pools and control proteins, in culture medium. Small intestinal biopsies from CD patients, both treated and untreated, and from controls, will be placed on steel mesh grids held above and just touching the culture media. The cultures will be kept at 37° C. overnight in an atmosphere of 5% CO₂ and 95% oxygen at two atmospheres pressure. Following 16 h culture, the tissue will be snap frozen and stored in liquid nitrogen prior to cutting frozen sections. Additionally, the culture media from the organ culture dishes will be stored for evaluation of interferon-gamma and interleukin 15 (IL-15) secretion using commercially available kits (R & D Systems Ltd, USA). These will be stained with haematoxylin and eosin and used to measure the enterocyte cell height (ECH), using a standard micrometer eyepiece and light microscopy, of at least thirty enterocytes as we have previously described (Shidrawi et al. 1995), (Biagi et al. 1999), (Martucci et al. 2003). Significant reductions in ECH following incubation with test peptide compared to negative control will be taken as a measure of celiac toxicity. Significant increases in IL-15 in supernatants of biopsies cultured with the test peptide compared to negative controls will also indicate toxicity. We will carry out approximately six tests using celiac small intestinal mucosa for each peptide or protein, with an equal number of controls. The results will be assessed by non-parametric statistical analysis. We will use these methods to confirm or exclude the toxicity of the gluten peptides and proteins, confirming the results of the T cell assays.

In vivo testing: The detoxified gluten proteins will be assessed using in vivo challenge studies in celiac patients as we have previously described. Briefly, these experiments involve instillation of the putative non-toxic protein into the duodenum of celiac patients over two hours. Duodenal biopsies are taken hourly over six hours. We had previously shown that examination of the morphological parameters from such biopsies including: (i) the ratio of villous height: crypt depth, (ii) enterocyte height and (iii) the number of intra-epithelial lymphocytes per 100 enterocytes taken together can be used as sensitive marker of in vivo gluten protein toxicity in celiac patients (Ciclitira et al. 1984b), (Sturgess et al. 1994), (Fraser et al. 2003), (Dewar et al. 2006). The resultant bread will also be tested in vivo in celiac patient volunteers using 6-week cross-over studies as previously described (Ciclitira et al. 1984b); (Ciclitira et al. 1984a).

### Cloning of Glutenin and Gliadin CD Epitopes

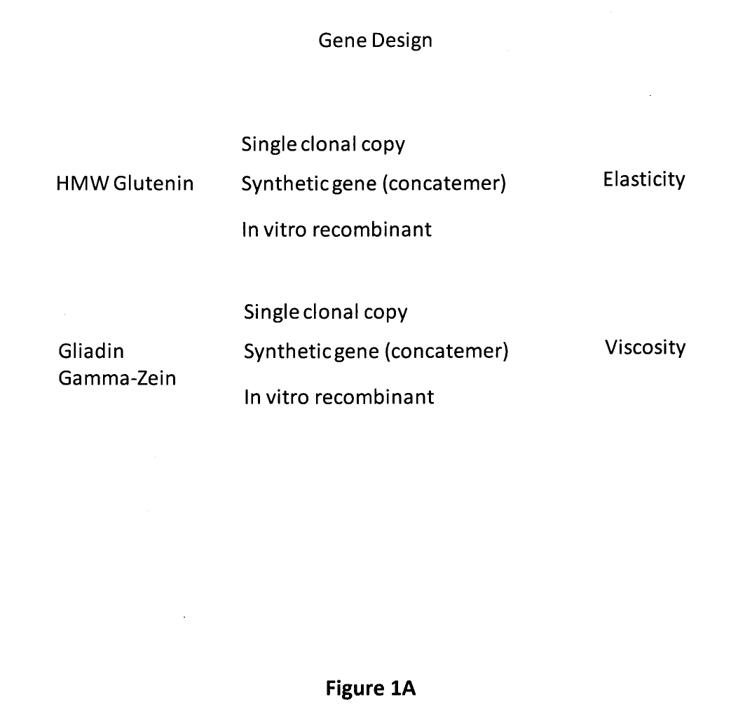

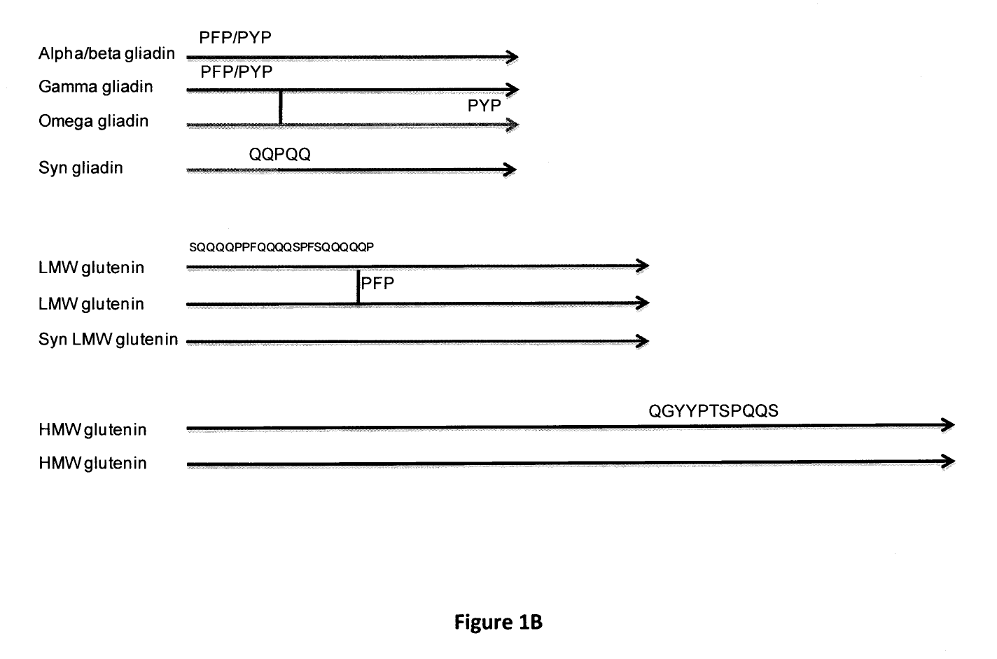

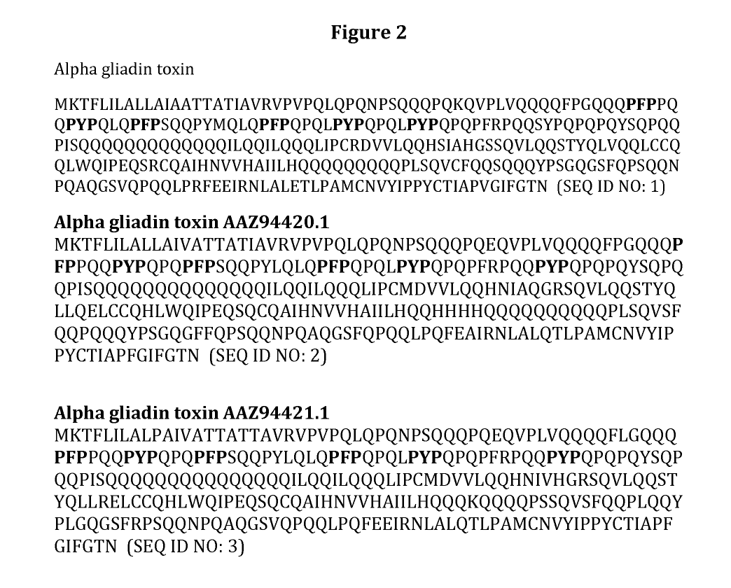

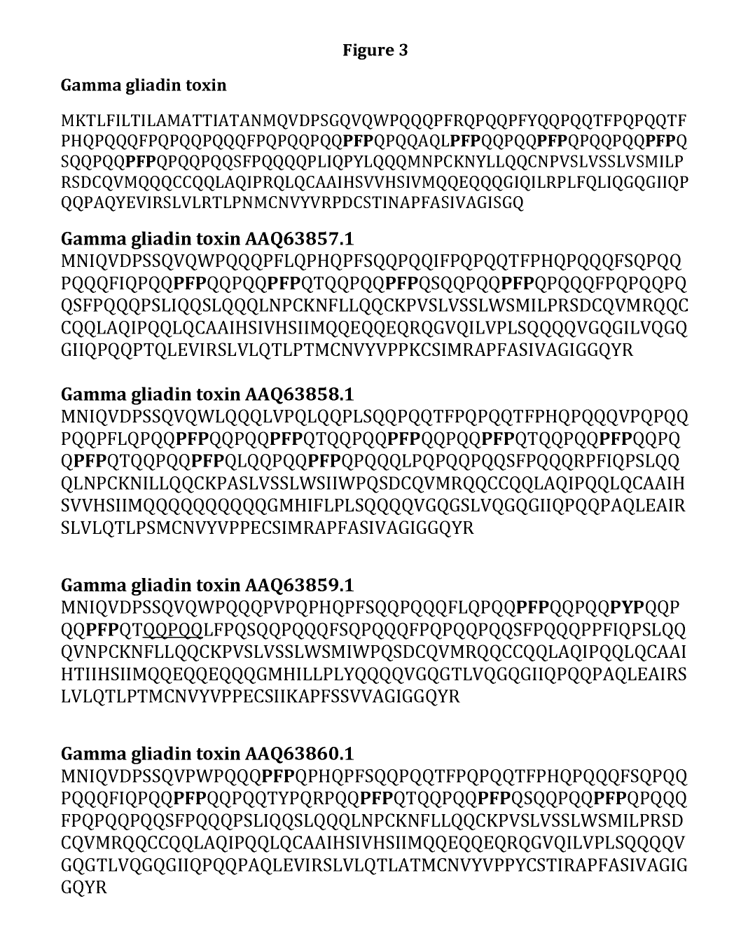

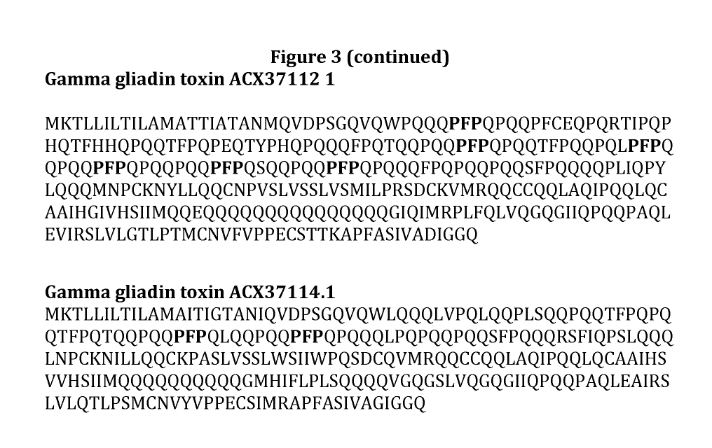

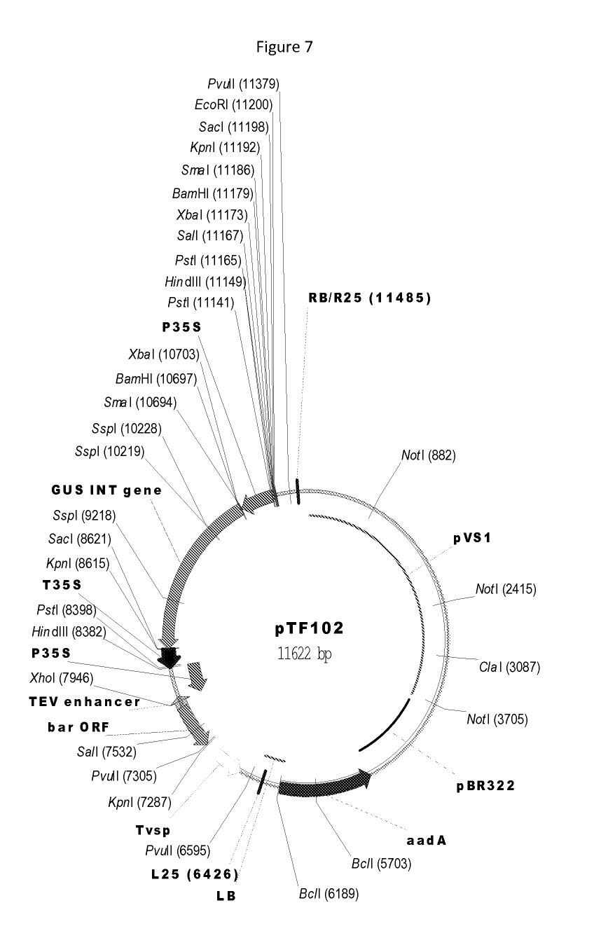

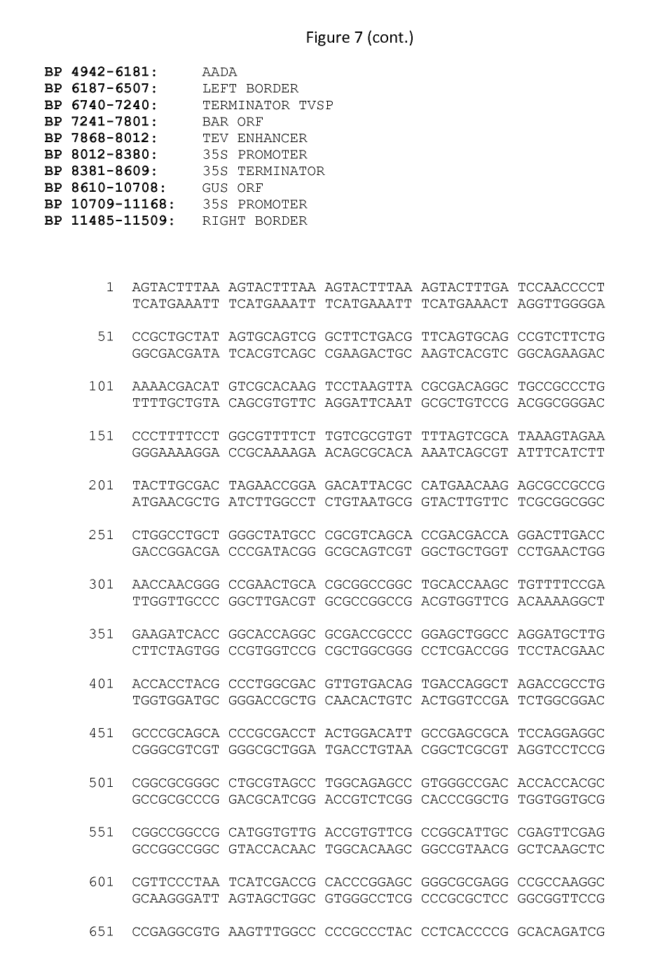

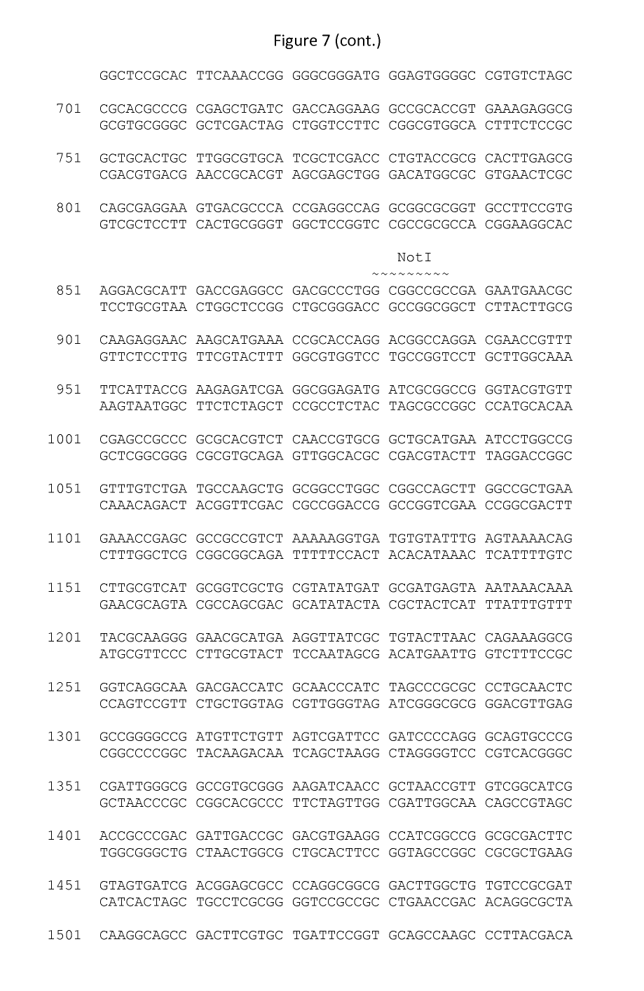

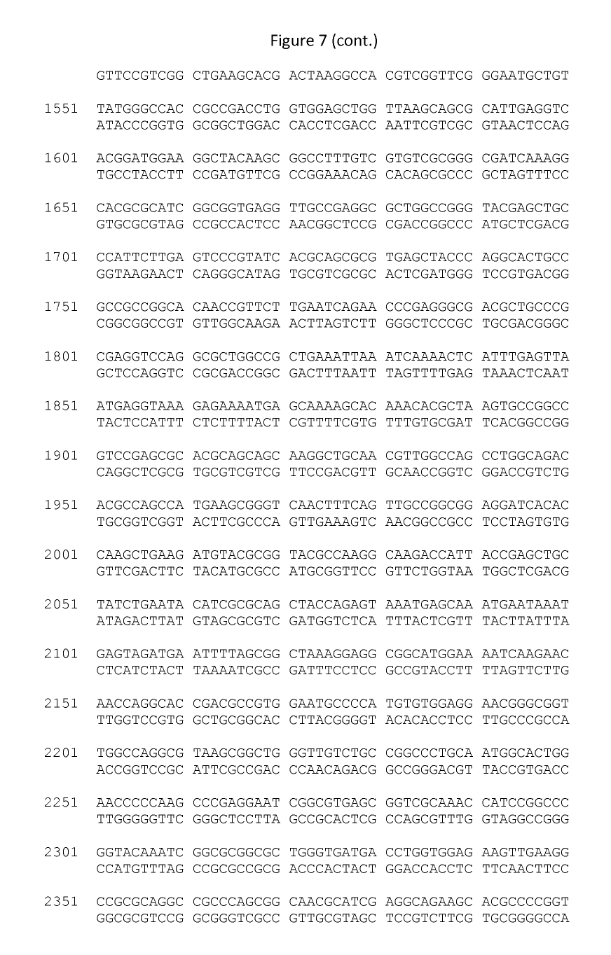

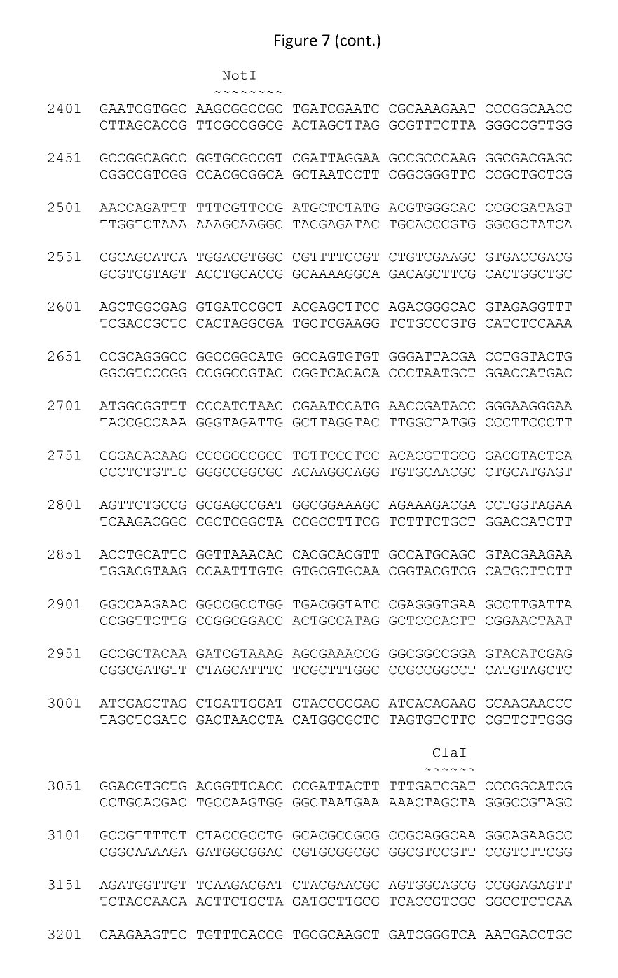

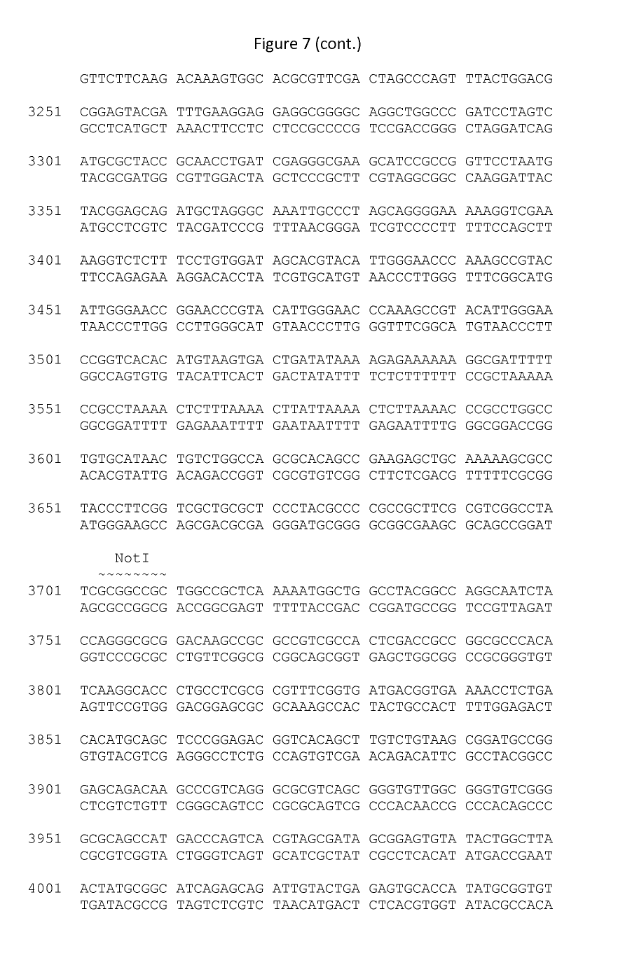

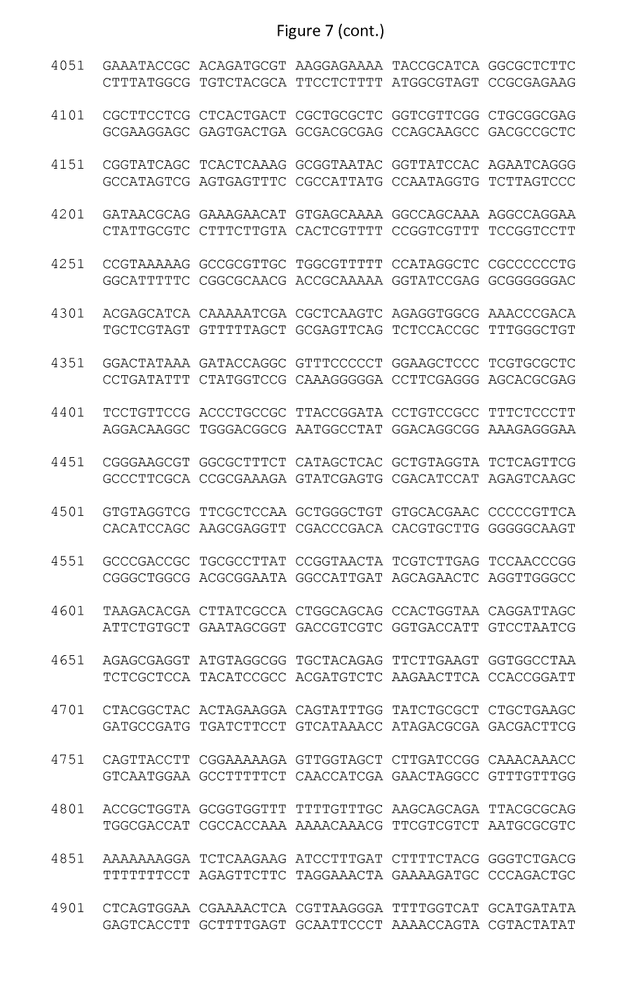

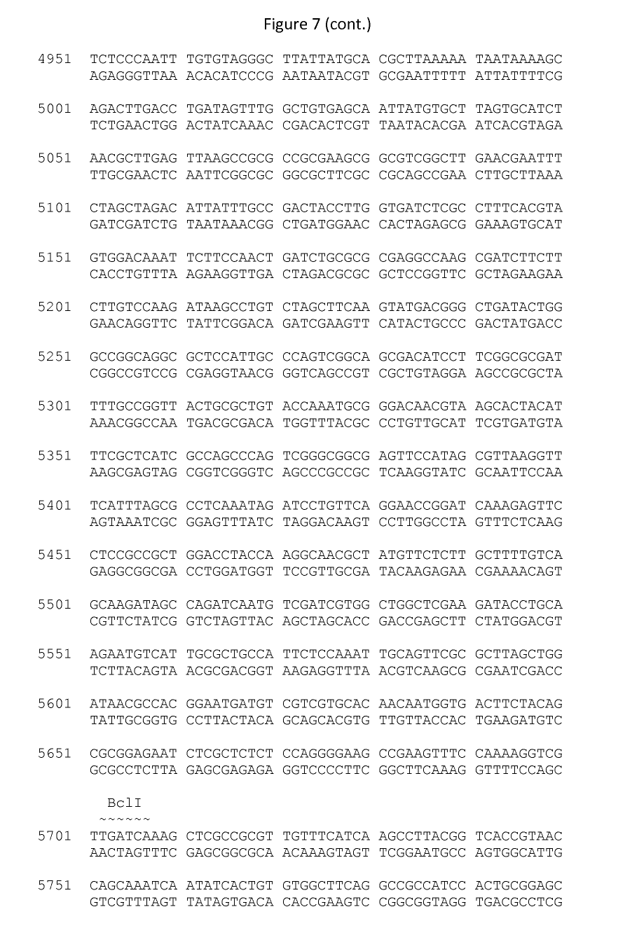

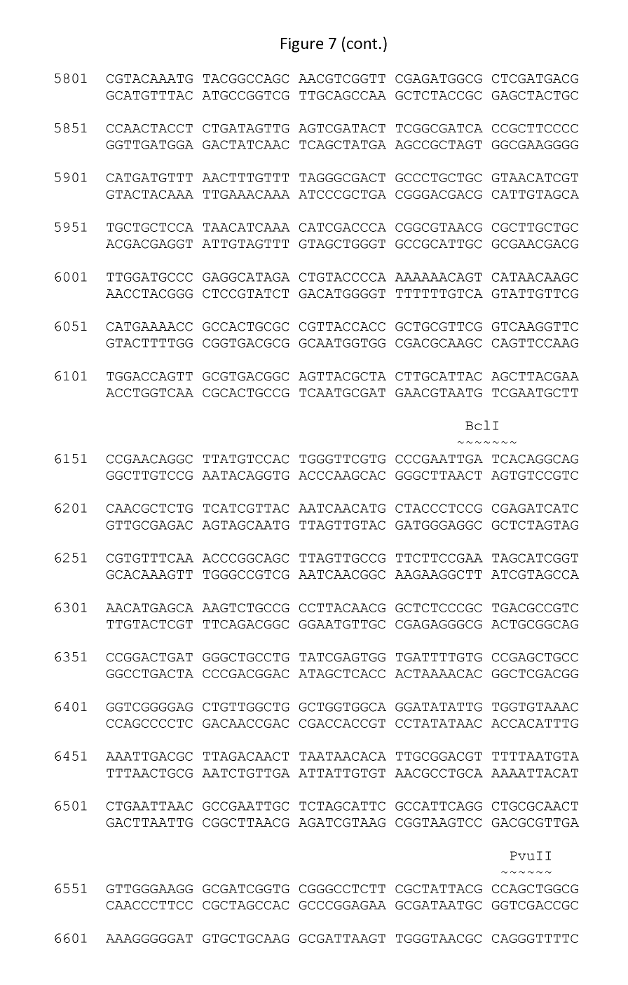

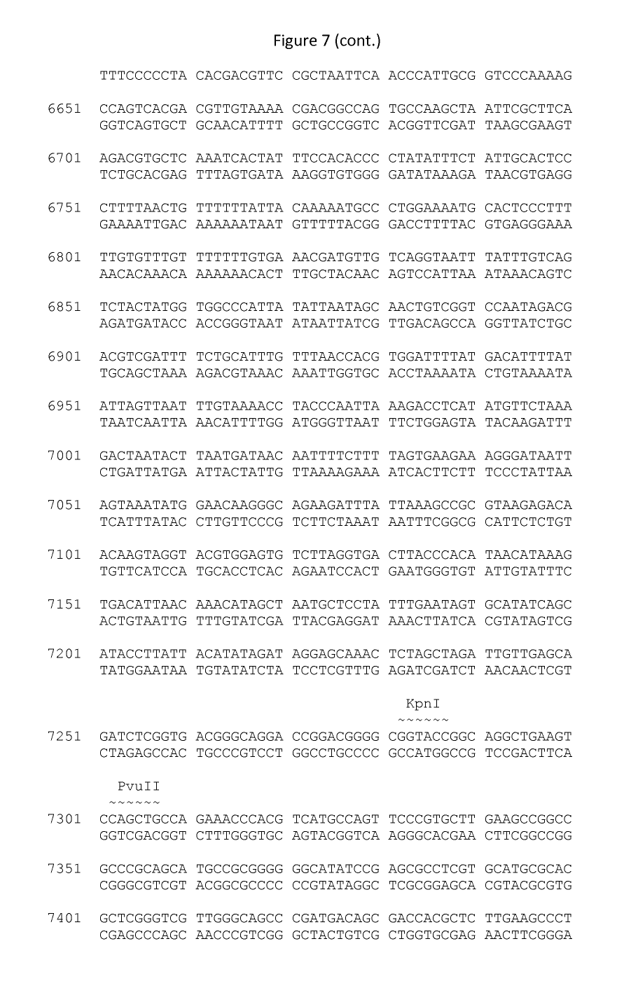

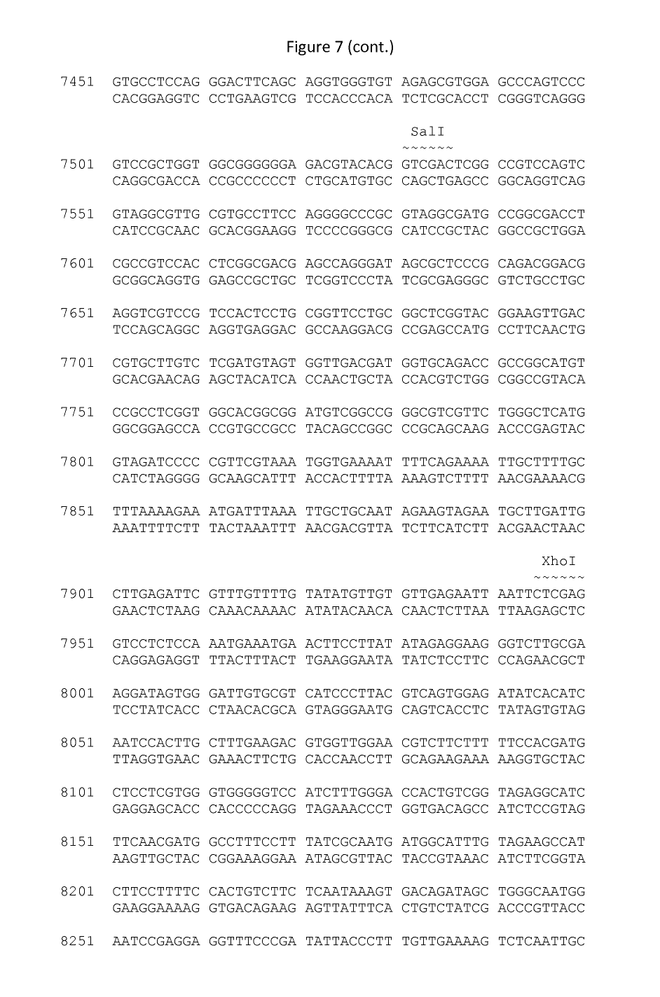

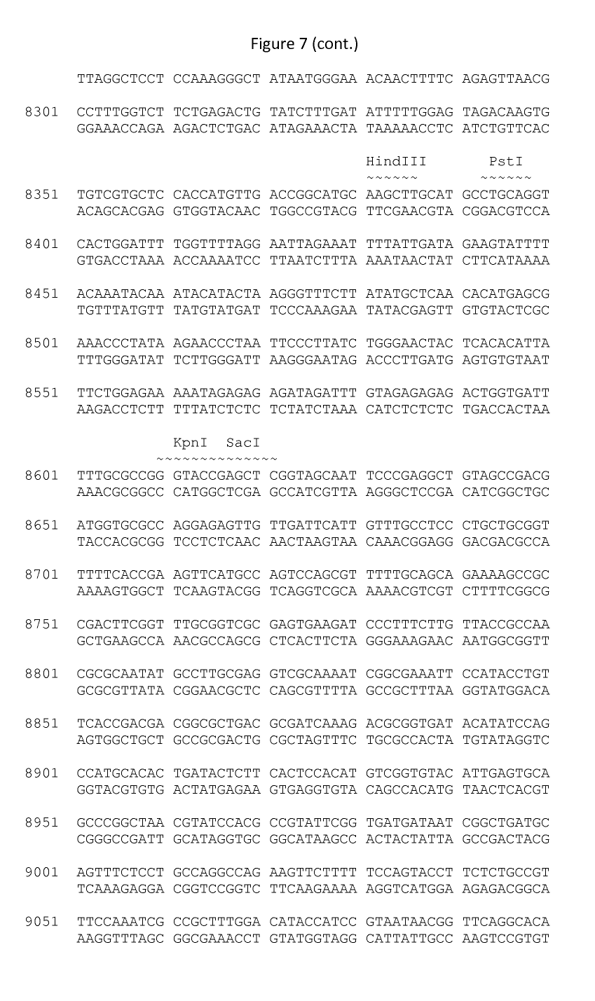

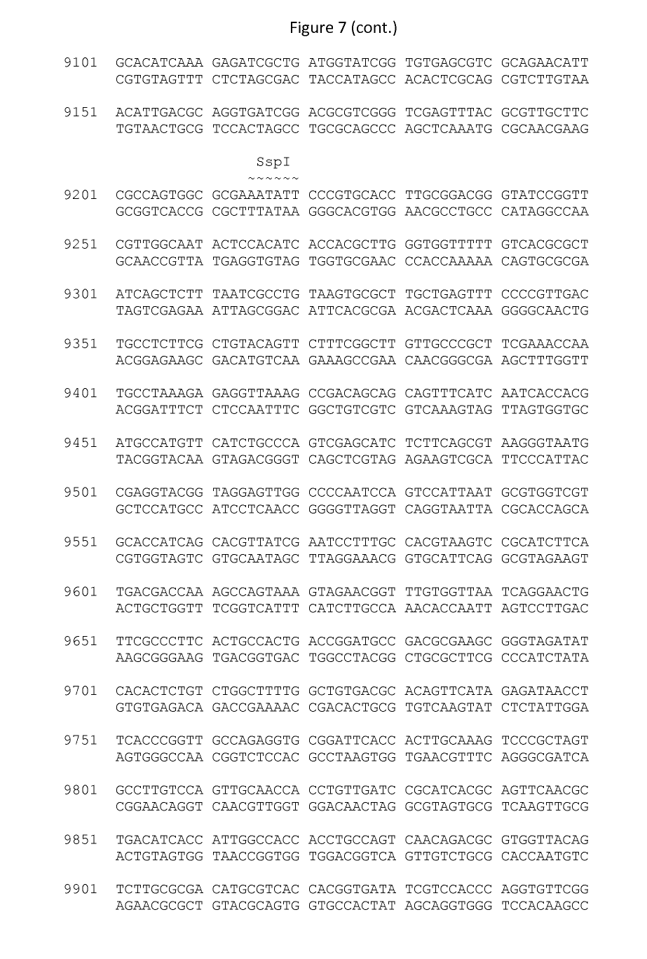

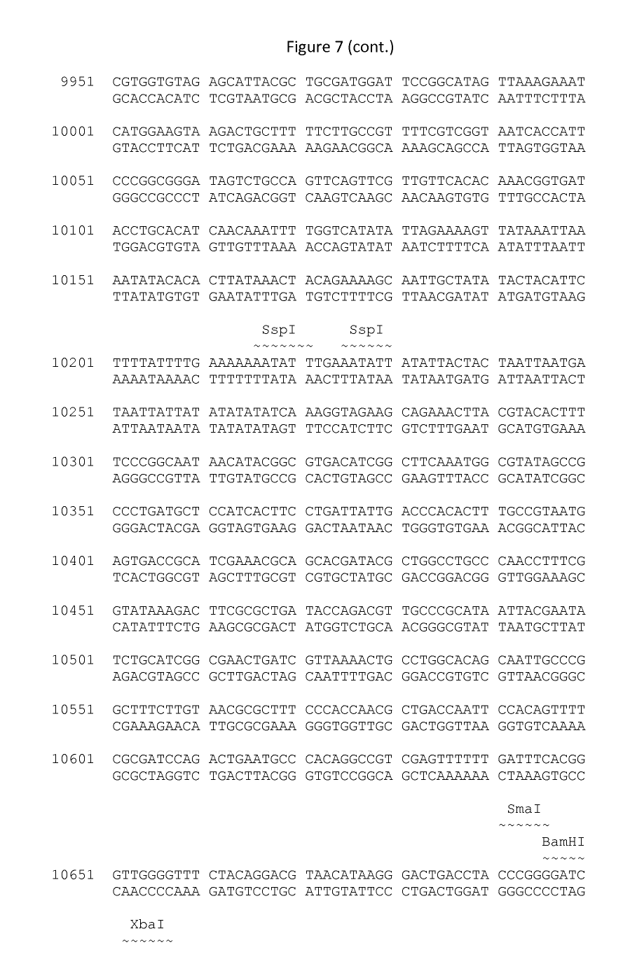

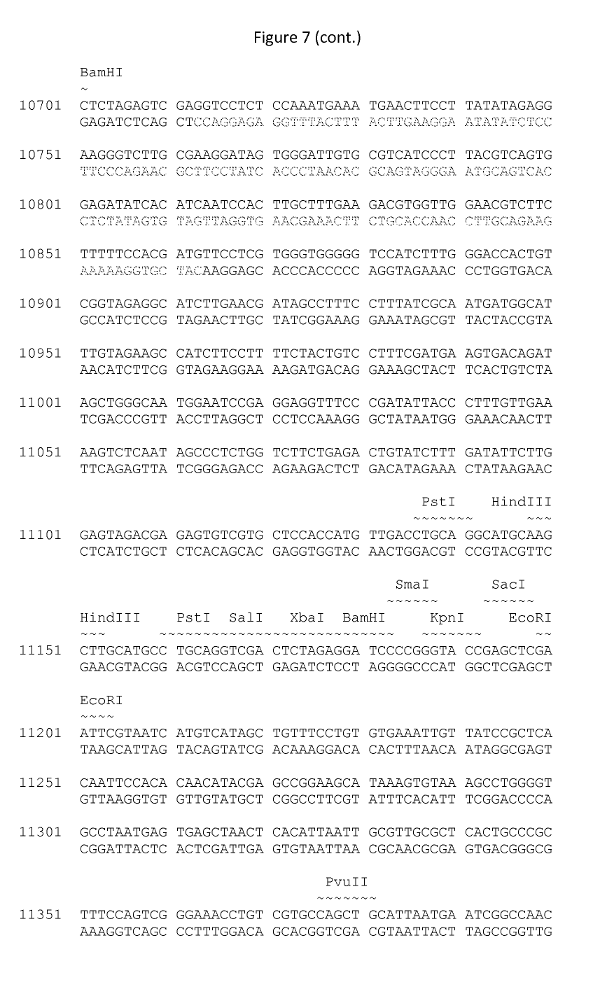

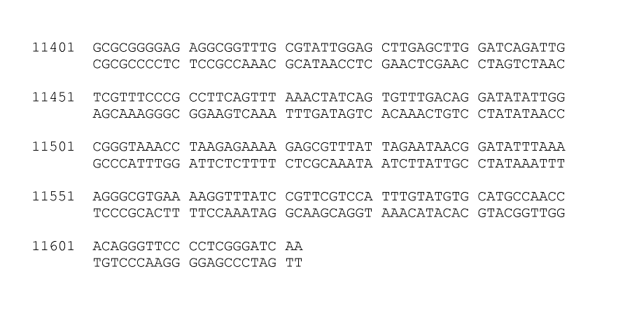

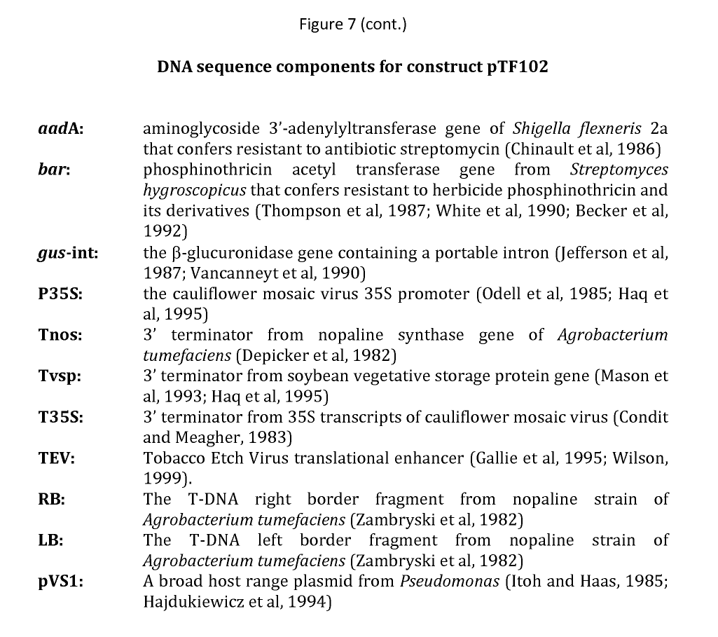

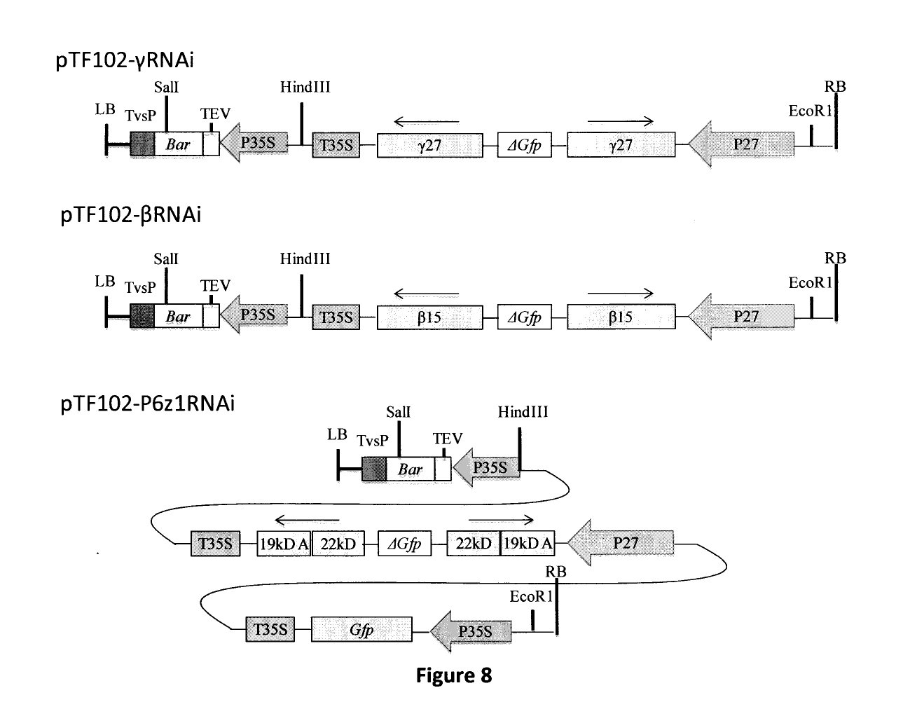

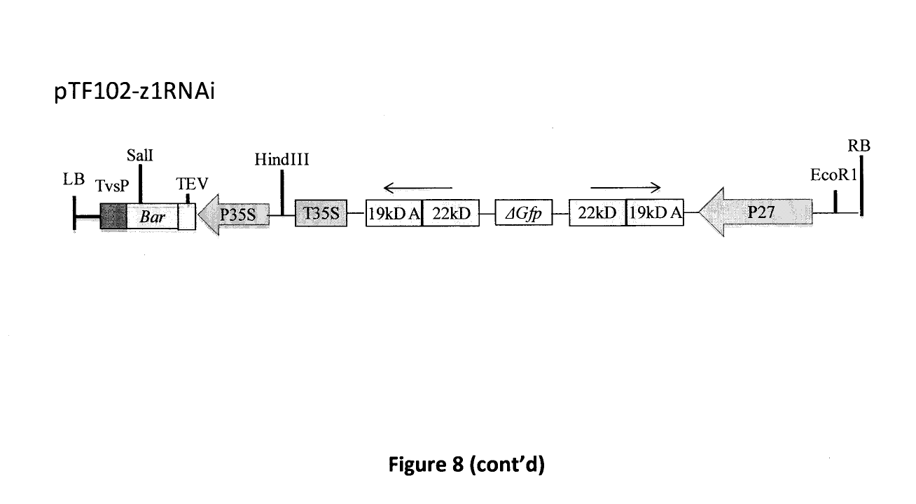

Generation of transgenes suitable for transformation in maize which lack CD causing epitopes: Based on natural variation, glutenin and gliadin genes will be selected, where different portions of the coding regions that are free of toxic epitopes. These will then be combined to form chimeric coding sequences that retain the physical properties of glutenins and gliadins, but do not cause the disease as shown in FIG. 1. These coding sequences will be sandwiched between a zein promoter such as the 27-kDa zein promoter and the 3′ end of zein gene to facilitate expression of synthetic glutenins and gliadins in maize endosperm as described previously (Wu et al. (2010) Proc. Natl. Acad. Sci. USA 107, 12810-12815). Synthetic genes will be inserted into the maize transformation vector pTF102 as shown in FIG. 7.

Expression of transgenes in maize and production of transgenic plants: Immature embryos of maize will be cultured as described previously (Wu et al. (2010) Proc. Natl. Acad. Sci. USA 107, 12810-12815). The maize transformation vector pTF102 carrying the synthetic glutenin and gliadin genes under a maize endosperm-specific expression system will be introduced into Agrobacterium for cocultivation with maize callus cells as described by Frame et al. cited in Wu et al. (2010) Proc. Natl. Acad. Sci. USA 107, 12810-12815. Transformed plant cells will be subjected to selection conditions following standard procedures as described previously. Plantlets will be regenerated and transferred to soil for further growth. Callus and early leaf samples will be subjected to PCR analysis to trace the transgene following standard procedures (Wu et al. (2010) Proc. Natl. Acad. Sci. USA 107, 12810-12815). After seed set, individual events will be monitored for expression by Western blot analysis. Transgenic seeds will be subjected to the bio-assay described above to confirm toxic-free synthesis of glutenin or gliading events.

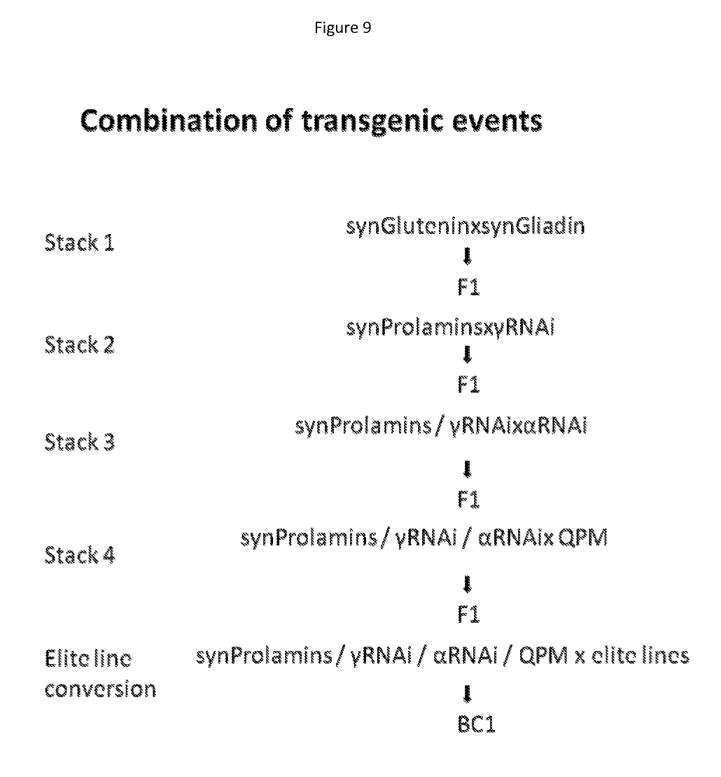

Expression of RNAi in maize for increasing production of glutens: Confirmed glutenin and gliadin events will be crossed as described in FIG. 9 (Stack 1). Expression of proteins will be analyzed using standard procedures like SDS PAGE and Western blot analysis. Stack 1 will also be confirmed to be toxic free. Additional crosses will be performed to generate stacks that exhibit increased glutenin and gliadin protein accumulation by replacing maize storage proteins in maize protein bodies. Electron microscopy will be used to monitor intact protein bodies as described previously (Wu et al. (2010) Proc. Natl. Acad. Sci. USA 107, 12810-12815). A synergistic effort will involve the combination of Quality Protein Maize (QPM) (Wu et al. (2010) Proc. Natl. Acad. Sci. USA 107, 12810-12815) and bakeability (stack 4). Each stack and parental lines will be subjected to rheological analysis as described below. Because transgenic events in the presence of different trans-acting RNAi constructs differ in expression levels and rheological qualities, a combination will be selected that will be best suited for bakeability.

The following examples are provided to illustrate certain embodiments of the invention. They are not intended to limit the invention in any way.

### EXAMPLE I

### Identification of CD-Associated Epitopes in Wheat Glutens

We have established gluten sensitive T-cell transformation and celiac small intestinal organ culture systems that are critical for the detection and validation of gluten containing compositions that do not induce CD and for the production of non-CD inducing flour.

Gluten sensitive T-cells will be obtained from small intestinal biopsies from autologous patients with either treated or untreated CD, as we previously described (Ellis et al. 2003). Briefly, small intestinal biopsies will be cultured with a peptic tryptic digest of wheat gluten (PT-glut). Collagenase will be used to disrupt the tissue and isolate the small intestinal lymphocytes that will be grown up with interleukin-2 The cells will be re-stimulated every seven days using peripheral blood mononuclear cells as antigen presenting cells, pre-incubated with PT-glut; the resultant T cell lines will be tested after 1-3 weeks for their responsiveness to tTG treated (i) PT-glut, which will serve as a positive control and to (ii) HMW-GS derived peptides or their analogues. The latter peptides are unlikely to need peptic tryptic digestion. The timing of the testing will be dependent on sufficient cell numbers being obtained. We will measure the relative stimulation index, SI, that is, comparison of the incorporation of tritiated thymidine by the T cells in the presence and absence of the test substance. A positive result is considered to be an SI of two with a significant difference (p<0.05) between the means of triplicate results. T cell culture supernatants will be analysed for interferon-gamma (IFN-γ) secretion using a commercially available kit (R&D system Ltd. USA) as a further marker of vitro toxicity.

### EXAMPLE II

### Generation of Transgenes Suitable for Transformation in Maize Which Lack CD Associated Epitopes and Identification of New Epitopes Via Pools of Synthetic Peptides

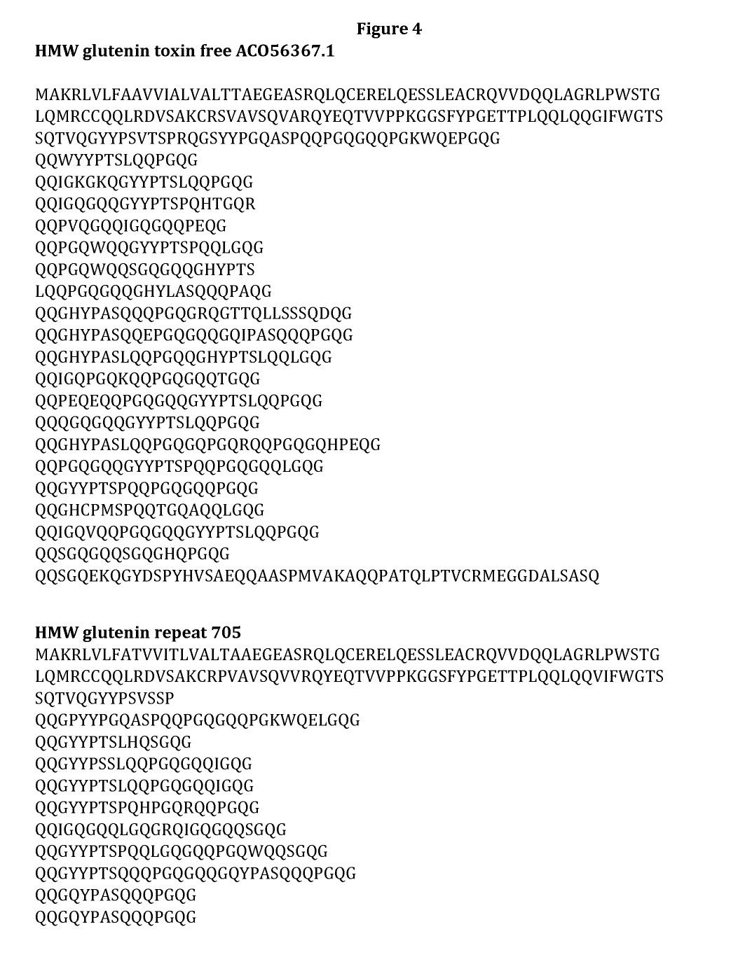

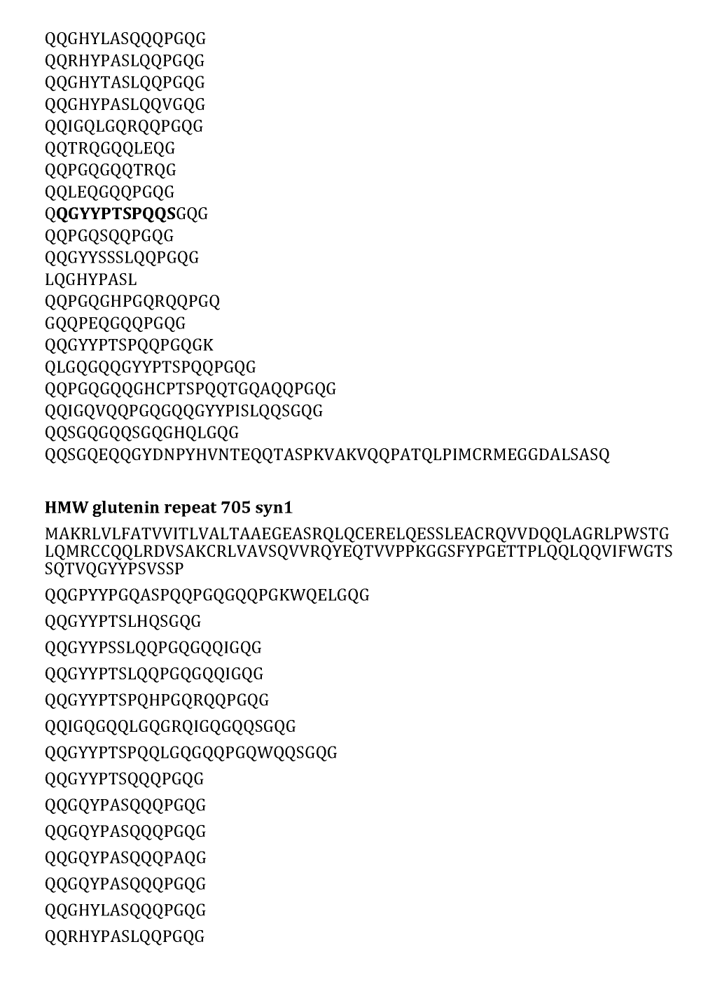

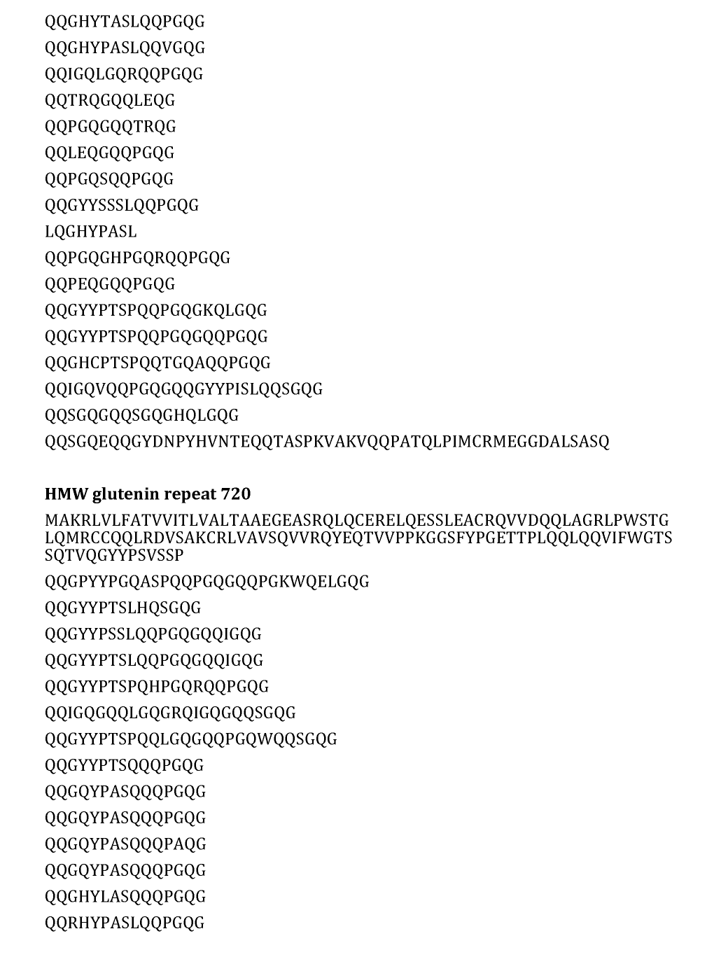

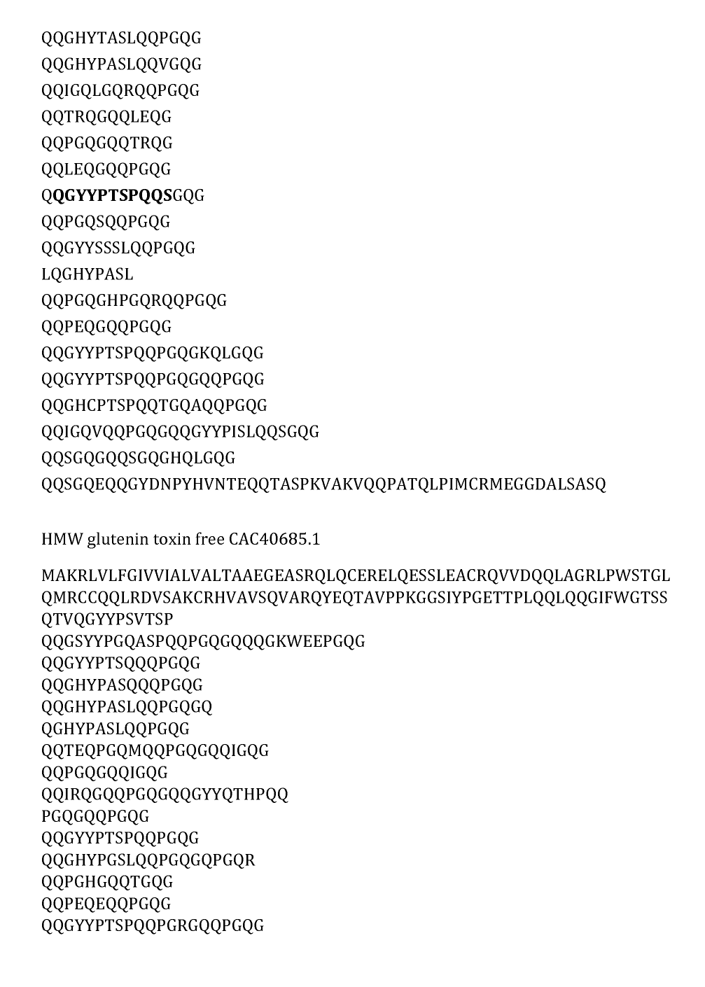

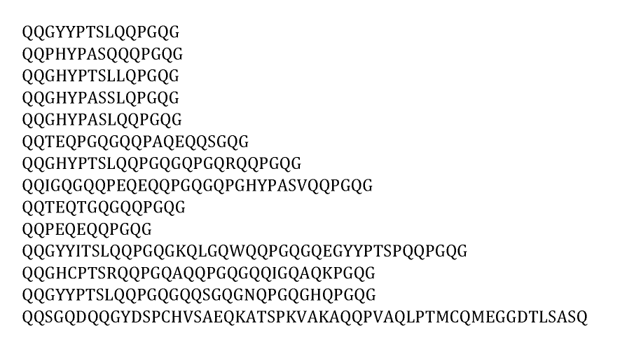

The challenge in overcoming CD is that traditional breeding cannot separate the hundreds of gene copies encoding a collection of proteins, which are very similar in structure but vary in their degree of CD toxicity. Furthermore, each protein comprises a 12-20 amino acid block that is tandemly repeated as shown in FIG. 4, producing variable peptide motifs of epitopes that can be either CD toxic or non-toxic. Therefore, synthetic genes consisting of non-toxic repeats that also provide the rheological properties of bakeable flour will be generated. The synthetic gene(s) can be introduced into a species other than wheat to avoid the presence of disease-triggering proteins. Because maize does not produce CD-triggering storage proteins and is cheaper to produce than wheat, it is the ideal platform for the development of consumable products that do not induce CD. In one approach we will transform maize with modified HMW1Dx5 and 1Dy10 and modified LMW gene sequences, by stacking transgenic events of synthetic gluten encoding gene sequences. We will also transform maize with DNA encoding gliadins modified to obviate celiac toxicity. We will also cross or stack transgenic events to complement the set of storage proteins required for the bread-making qualities of wheat.

Certain amino acid repeats that produce gluten protein epitopes that are CD toxic have been previously identified. Thus, we can create new variants that omit such epitopes. For example, HMW glutenins containing the QGYYPTSPQQS motif have been found to be toxic. However, natural variants of this gluten exist in nature that are free of this motif (see FIG. 4).

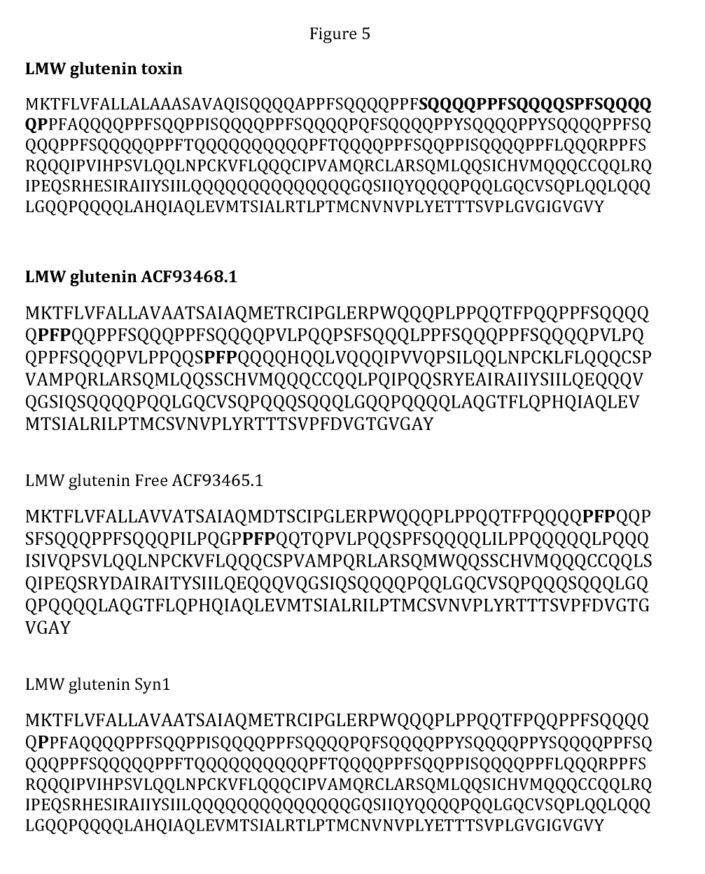

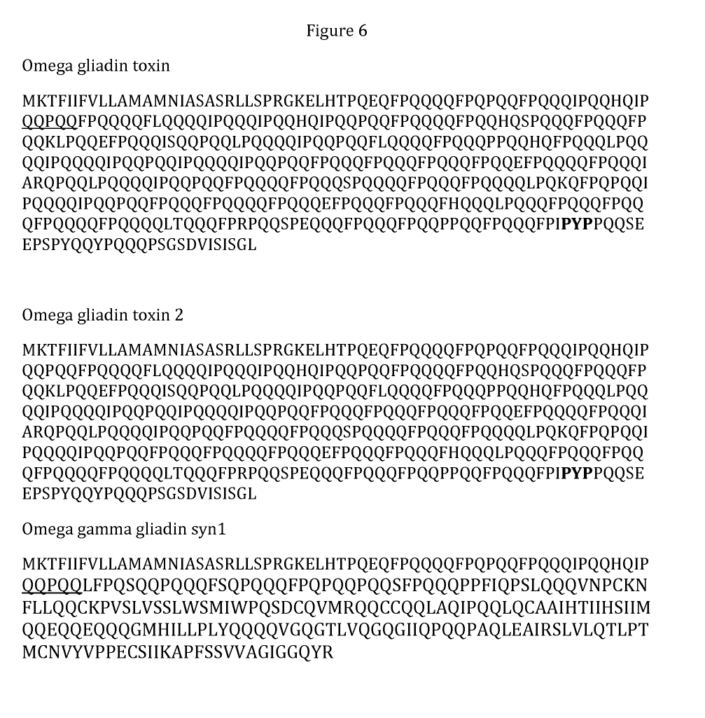

LMW glutenins might contain the SQQQQPPFSQQQQSPFSQQQQQP or PFP motifs that are toxic, but one can recombine in vitro the first 56 amino acids with the last 296 amino acids of two different LMW glutenins to gain a hybrid that is free of either motif (see FIG. 5). In respect to the gliadins, multiple PFP and PYP motifs can be detected that are believed to be toxic. An in vitro recombinant of two gliadins via the common QQPQQ sequence would provide the first 61 amino acids from an omega gliadin and the last 197 amino acids from a gamma gliadin to produce a hybrid gliadin free of the known toxic epitopes (see FIG. 5).

Wheat gliadins and glutenins will be further assessed in order to ensure all toxic epitopes have been identified. It is possible that the chosen recombinant glutenins and gliadins contain as yet unknown toxic epitopes. To avoid these, we can employ a more systematic approach to characterize all natural variable epitopes. For example, in Ae. tauschii genome, which is a model for the D genome of wheat, the Anderson lab in California has found eight omega, eight gamma, and 5 LMW glutenins clustered within a short interval (PAG XX Poster, 2012). Of these 21 genes, about 15 are expressed. In hexaploid wheat we could have three times as many. In wheat, four to five out of 6 HMW glutenin genes are expressed (Gu et al. 2006). In total there could be about 50 expressed genes in wheat, excluding a few pseudogenes. If we were to include 12 cultivars, we could have about 600 different allelic gene copies. Because each of those has multiple variable epitopes, we could have up to a few thousand variable epitopes. On the other hand, 90% of them could be clustered into common motifs. In other words, they may be only a few hundred variable motifs. Currently, about 31 motifs have been classified as CD immuno-stimulatory (Sollid et al. 2012). Only one of those per molecule would be sufficient to make gluten intolerable for CD patients. If there are in addition to these 31 know motifs additional ones that need to be removed, we will have all common motifs synthesized as synthetic peptides and test them in pools of ten until all are identified.

To determine the exact number of variable motifs, we will sequence the expressed glutenin and gliadin genes from a dozen cultivars. We will design universal primers (sequences conserved within the coding regions of each subfamily (Shewry and Halford 2002) for cDNA synthesis of nearly full-length cDNAs. Because many gene copies have pre-mature stop codons the use of mRNA selects only expressed genes (Dong et al. 2010). Wheat prolamins represent more than 50% of all endosperm proteins. Therefore, mRNA of immature wheat endosperm does not need to be enriched for prolamin mRNA. Furthermore, the use of specific synthetic primers spares us the need for PCR amplification and size selection. Only first-strand synthesis long enough to reach the 5′ end of mRNAs, will be complementary to the reverse primer for second-strand synthesis. Incomplete first strands would stay single-stranded and be lost in the cloning procedure. PCR amplification could produce artifacts of chimeric mRNAs in particular because of the tandem repeat in the center portion of the coding regions. In fact, no PCR or cloning will be performed before the construction of sequencing libraries. Double-stranded DNA from the different glutenin and gliadin groups will be fragmented into small fragments of about 200 base pairs using DNAse I. Each fraction will be bar-coded so that we can assign them to each glutenin and gliadin group after sequencing computationally. We will sequence the bar-coded libraries using a next generation sequencing platform with SOLiD 5500s, which with mate pairs can sequence 60 by from each end with a depth of 10 GB per run. Sequences will be catalogued and the complexity of each library assessed. Because of the enormous sequence coverage and known gene sequences of each class of prolamins, sequence reads longer than the peptide repeats, and the use of mate-pairs, will be used for the reconstruction of intact mRNA sequences. We have used RNAseq for differential expression of microRNAs and are familiar with such redundant datasets (Calvino et al. 2011).

After reconstruction of the expressed genes, we will computationally cluster all conserved epitopes. Because of the redundancy of certain motifs, we estimate that in total not more than a few hundred variable epitopes will be catalogued. These will serve as a guide synthesizing the encoding peptides. We could optionally have glutamines replaced with glutamic acid and thereby avoiding the tTG step described above. By pooling ten peptides at a time, we could test those pools in our in-vitro cell assays. By eliminating pools that are non-toxic, we can narrow down those motifs that are positive in our cell assays. With reiteration, we will able to test individual peptide-candidate toxic epitopes in our in-vitro cell assays. This should result into a comprehensive collection of toxic epitopes beyond what it is now known (Sollid et al. 2012).

These will be compared to the collection of expressed genes. We then can select those that are free of toxic epitopes. We will also repeat the introduction of natural non-recombinant glutenin and gliadins into maize for confirmation in our in-vitro assays.

Although it is feasible to use site-directed mutagenesis to alter amino acid motifs to be CD toxic epitope free, our approach has the advantage that motif variants already exist in nature and are probably more stable than those test-tube derived. Synthetic genes can then be assembled consisting of a mosaic of fragments of naturally occurring amino-terminal, central, and carboxy-terminal regions that are CD toxic epitope free but which mimic the rheological properties of wheat gluten. To aid in the design of CD non-toxic gluten, we will also test the potential of maize storage proteins to produce increased viscosity from its related storage proteins, the gamma zeins. Here we will use a two-pronged approach. We will analyze the composition of storage proteins of maize lines that have been used to generate palatable maize-based gluten-free bread (Portuguese gluten-free broa) and specific RNAi lines where different classes of maize storage proteins are silenced. For instance, in the first case one class could be shown to increase viscosity and in the second to decrease viscosity. Such knowledge could also be used to achieve a combinatorial effect of synthetic and natural genes to fine tune parameters of viscosity and elasticity without CD toxic epitopes.

### EXAMPLE III

### Expression of Transgenes in Maize and Production of Transgenic Plants

In a preferred approach the transgenes of the invention will be expressed in a tissue-specific manner. The storage proteins in maize accumulate in the endosperm of the seed and are compartmentalized in subcellular structures, called protein bodies (PB). The new CD-free constructs will be used to transform maize embryogenic cultures by an Agrobacterium-mediated method (Frame et al. 2002). Maize transformation using the particle bombardment method (Lai and Messing 2002; Segal et al. 2003; Song et al. 2004) and the Agrobacterium method (Wu et al.

2010; Wu and Messing 2009; Wu and Messing 2010) will be employed. We have succeeded high-level of expression of transgenes with the 27-kDa and the 10-kDa zein promoters. For proper processing of wheat synthetic glutens into protein bodies, it also will be necessary to equip transgenes with a maize signal peptide. We will transform our B73/A188 hybrid as the primary target. We will grow the first generation transgenic plants and harvest seed therefrom. After cocultivation with Agrobacterium, transformed embryos will be selected on medium containing the selectable marker. Regenerated plantlets will be ultimately transferred to soil following standard procedures. Transgenic plants will be crossed with the different RNAi lines for optimal gene expression. In an alternative approach, we can assemble constructs with all modified gluten genes at the same time. We will pursue this approach once we have explored the immunological properties of the new proteins individually. A single multigene construct should facilitate the commercial applications resulting from this research.

### EXAMPLE IV

### Expression of RNAi in Transgenic Maize

The expression of synthetic gluten transgenes by crossing the primary transformation events can be tested with three types of maize knock-down lines (Wu, Y. and Messing, J. (2010) Plant Physiol. 153, 337-347). Two lines represent the knock-down of the maize gamma and beta prolamins. They are cysteine-rich and would be the closest substitution for the wheat gluten from an evolutionary point of view. It also has been reported that gamma zeins expose potential allergens (Krishnan et al J Agr Food Sci 2010), which could be avoided at the same time. The second concerns the bulk of prolamins in maize, also called the alpha zeins. We now have a maize transgenic RNAi line developing against both the 19 and 22 KDa alpha zeins from maize, which is expected to be more efficient than our earlier one against the 22-kDa zeins alone (Segal et al Genetics 2003). We had shown that reduction of alpha zein by RNAi can increase the lysine content in maize, which would add a great benefit for the use of maize with greater nutritional value. Furthermore, combining the alpha and gamma zein RNAi with the Illinois High Protein trait could result in the enhanced expression of wheat prolamins in maize (WU and MESSING 2012). Because RNAi transgenic lines are dominant, it will be easy to test the properties of different storage proteins in respect to expression levels, bread-making abilities, CD-free epitopes, and nutritional qualities. We will confirm that the resulting maize flour, which contains all the necessary wheat seed storage proteins, and lacking the respective zeins, will have comparable rheological properties to wheat flour and will therefore be bakeable into palatable bread. The novel flour could be used for a variety of baking, thickening and other culinary purposes associated with wheat flour.

### EXAMPLE V

Rheological Properties of Flour Produced from Transgenic Maize Expressing Recombinant Wheat Glutens

The four different recombinant proteins will be expressed in maize, and then the effects of one or more of these proteins on the baking properties of the resultant flour will be assessed. The maize grain kernels are removed from the cob and are then milled in a suitable metal headed mill. The product is then separated with a two to five hundred size micron sieve into the constituent parts comprising the flour and the ground kernels. This enables production of the milled flour, which is suitable for analytical testing and baking purposes. The extensibility and elasticity of wheat flour depends not only on the physical properties of the HMW-GS to entrap carbon dioxide but also on the gliadins to slide over one another. We suggests that maize zeins probably have characteristics similar to gliadins which may enable maize flour containing the detoxified 1Dx5 and 1Dy10HMW-GS to produce a dough that will prove satisfactory for generating bakeable bread.

We have undertaken preliminary baking experiments with maize meal to which we added gluten. We used 90% maize meal with 10% gluten, which was found to have palatable bread with good crust. Wheat contains approximately 10% of gluten.

We anticipate that increasing expression efficiency in maize with RNAi technology should permit the generation of sufficient amount of celiac detoxified wheat gluten to provide adequate rheological and sensory characteristics to generate palatable bread. We will also assess different maize cultivars for their suitability in baking experiments. Brites et al (BRITES et al. 2008) reported that the cultivars Fandango, a regional Dent type and Pigaro, a regional Flint type of maize were suitable to produce maize-based, gluten-free bread. They used processing parameters to study sensory and instrumental quality. They concluded that the Fandango strain was more suitable for gluten-free bread production. We will take hemi- or homozygous transgenic maize plant cultivars that contain one to four wheat seed storage protein genes with concomitant down-regulation of α-zein genes as the basis for the initial investigation of the rheological and baking properties of the resultant flour. This will be a prelude to assessing the sensorial qualities of the resultant bread by an experienced sensory panel. Following sensory assessment of the resultant bread, should modification be required we will utilize previously described methods to improve the baking and sensory characteristics of the final product with agents such as addition of colloids (BRITES et al. 2008).

Abrogation of toxicity of the resultant bread can be confirmed in vivo clinical toxicity studies in celiac subject volunteers. The foregoing approach will not only provide a significant improvement in the available treatment of CD but will also provide an improved dietetic adjunct for other conditions for which a gluten free diet is taken, for example, irritable bowel syndrome. Product testing will be undertaken with cross over studies in 10-20 patients over six weeks with endoscopy and small intestinal biopsy before and after the test period, to confirm lack of celiac toxicity of bread made from the non-toxic modified maize flour described herein.

### REFERENCES

1. Anderson R, Degano P, Godkin A, Jewell D, Hill A. (2000). In vivo antigen challenge in celiac disease identifies a single transglutaminase-modified peptide as the dominant gliadin T cell epitope. Nat Med 6: 337-342.
2. Anderson, O D and Greene, F C (1989). Nucleotide sequence of Glu-D1-2b a high molecular weight glutenin gene from the D-Genom of a hexaploid bread wheat. Nucleic Acids Res. 17: 461-462.
3. Arentz-Hansen H, Korner R, Molberg O, et al. (2000). The intestinal T cell response to alpha-gliadin in adult celiac disease is focused on a single deamidated glutamine targeted by tissue transglutaminase. J Exp Med 191: 603-612
4. Arentz-Hansen H, McAdam S, Molberg O. (2002). Celiac lesion T cells recognize epitopes that cluster in regions of gliadins rich in proline residues. Gastroenterol 123: 803-9.
5. Biagi, F., H. Ellis, N. Parnell, R. Shidrawi, N. O'Reilley et al. (1999). A non toxic analogue of a coeliac-activating gliadin peptide: A basis for immunomodulation? Gut 44: A77-A77.
6. Biagi F, Ellis H J, Parnell N D J, Shidrawi R G, Ciclitira P J (1999). A Non-Toxic Analogue Of A Celiac Activating Gliadin Peptide: A Basis For Immunomodulation. Alimentary Pharmacology and Therapeutics 13: 945-950
7. Brites, C. M., C. A. L. dos Santos, A. S. Bagulho and M. L. Beirao-Da-Costa (2008). Effect of wheat puroindoline alleles on functional properties of starch. European Food Research and Technology 226: 1205-1212.
8. Calvino, M., R. Bruggmann and J. Messing (2011). Characterization of the small RNA component of the transcriptome from grain and sweet sorghum stems. BMC Genomics 12: e356.
9. Ciclitira, P. J., H. J. Ellis and N. L. K. Fagg (1984). Evaluation of a Gluten Free Product Containing Wheat Gliadin in Patients with Celiac-Disease. British Medical Journal 289: 83-83.
10. Ciclitira, P. J., D. J. Evans, N. L. Fagg, E. S. Lennox and R. H. Dowling (1984). Clinical testing of gliadin fractions in coeliac patients. Clin Sci (Loud) 66: 357-364.
11. Ciclitira P, Evans D, Fagg N, Lennox E, Dowling R. (1984). Clinical testing of gliadin fractions in celiac patients. Clin Sci 66: 357-364.
12. Corrao G, Corazza G R, Bagnardi V et al (2001). Mortality in patients with celiac disease and their relatives: a cohort study. Lancet. 358 (9279): 356-361.
13. De Ritis 1984, Dewar D, Amato M, Ellis H, Pollock E, Gonzales-Cinca N, Wieser H, Ciclitira P (2006). Immunogenicity and in vivo toxicity of HMW glutenins in celiac disease. Eur J Gastroenterol Hepatol 18: 483-491.
14. Dewar, D. H., M. Amato, H. J. Ellis, E. L. Pollock, N. Gonzalez-Cinca et al. (2006). The toxicity of high molecular weight glutenin subunits of wheat to patients with coeliac disease. Eur J Gastroenterol Hepatol 18: 483-491.
15. Dong, L., X. Zhang, D. Liu, H. Fan, J. Sun et al. (2010). New insights into the organization, recombination, expression and functional mechanism of low molecular weight glutenin subunit genes in bread wheat. PLoS One 5: e13548.
16. Ellis H, Pollock E, Engel W, Fraser J, Rosen-Bronson S, Wieser H, Ciclitira P (2003). Investigation of the putative immunodominant T cell epitopes in celiac disease. Gut 52: 212-217.
17. Frame, B. R., H. Shou, R. K. Chikwamba, Z. Zhang, C. Xiang et al. (2002). Agrobacterium tumefaciens-mediated transformation of maize embryos using a standard binary vector system. Plant Physiol 129: 13-22.
18. Fraser J S, Engel W, Ellis H, Moodie S, Pollock E, Wieser H, Ciclitira P (2003). Celiac disease In-vivo toxicity of the putative immunodominant epitope. Gut 52: 1698-1702.
19. Gu, Y. Q., J. Salse, D. Coleman-Derr, A. Dupin, C. Crossman et al. (2006). Types and rates of sequence evolution at the high-molecular-weight glutenin locus in hexaploid wheat and its ancestral genomes. Genetics 174: 1493-1504.
20. King A, Ciclitira P. (2000). Celiac disease: strongly heritable, oligogenic, but genetically complex. Mol. Genet. Metab. 71: 70-75.
21. Kasarda D, Okita T, Bernardin J et al. (1984). Nucleic acid (cDNA) and amino acid sequences of alpha-type gliadin from wheat (Triticum aestivum). Proc Natl Acad Sci USA 81: 4712-4716.
22. Kieffer R, Wieser H, Henderson M H, Graveland A (1998). Correlation of the breadmaking performance of wheat flour with rheological measurements on a micro-scale. J Cereal Sci 27: 53-60.
23. Lai, J., and J. Messing (2002). Increasing maize seed methionine by mRNA stability. The Plant Journal 30: 395-402.
24. Langridge, P. and Feix, G. (1983). A zein gene of maize is transcribed from two widely separated promoter regions. Cell 34: 1015-1022.
25. Martucci S, Fraser J S, Biagi F, Corazza G, Ciclitira P, Ellis H. (2003). Characterizing one of the DQ2 candidate epitopes in celiac disease: A-gliadin 51-70 toxicity assessed using an organ culture system. Eur J Gastroenterol Hepatol 15: 1293-1298.
26. Mazzarella et al. (2003). An immunodominant DQ8 restricted gliadin peptide activates small intestinal immune response in vitro cultured mucosa from HLA-DQ8 positive but not HLA-DQ8 negative celiac patients. Gut 52(1): 57-62.
27. Mitea, C., E. M. Salentijn, P. van Veelen, S. V. Goryunova, I. M. van der Meer et al. (2010). A universal approach to eliminate antigenic properties of alpha-gliadin peptides in celiac disease. PLoS One 5: e15637.
28. Muller S, Wieser H (1997). The location of disulphide bonds in monomeric y-type gliadins. J Cereal Sci 26: 169-176
29. Nassef H M, Bermundo Redondo M C, Ciclitira P J, Ellis H J, Fragoso A, O'Sullivan C K (2008). Electrochemical immunosensor for detection of celiac disease toxic gliadin in foodstuff. Anal Chem 80: 9265-9271.
30. Payne P I, Nightingale M A, Krattinger A F, Holt L M (1987). The relationship between HMW glutenin subunit composition and the bread-making quality of British-grown wheat. J Sci Food Agric 40: 51-65.
31. Segal, G., R. Song and J. Messing (2003). A new opaque variant of maize by a single dominant RNA-interference-inducing transgene. Genetics 165: 387-397.
32. Shidrawi R, Day P, Prezemioslo R, Ellis H, Nelufer J, Ciclitira P (1995). In vitro toxicity of gluten peptides in celiac disease assessed by organ culture. Scand J Gastroenterol. 30: 758-763.
33. Shewry, P. R., and N. G. Halford (2002). Cereal seed storage proteins: structures, properties and role in grain utilization. J Exp Bot 53: 947-958.
34. Shidrawi, R. G., P. Day, R. Przemioslo, H. J. Ellis, J. M. Nelufer et al. (1995). In vitro toxicity of gluten peptides in coeliac disease assessed by organ culture. Scand J Gastroenterol 30: 758-763.
35. Silano M, Volta U, Vincenzi A D, Dessi M, Vincenzi M D (2008). Effect of a gluten diet on the risk of enteropathy-associated T-cell lymphoma in celiac disease. Dig, Dis Sci 53(4): 972-976.
36. Sollid L, Khosla C (2005). Future therapeutic options for celiac disease. Nat Clin Pract Gastroenterol Hepatol. 2: 140-147.
37. Sollid et al. (2012). Nomenclature and listing of celiac disease relevant gluten T-cell epitopes restricted by HLA-DQ molecules. Immungenetics epub ahead of print.
38. Song, R., G. Segal and J. Messing (2004). Expression of the sorghum 10-member kafirin gene cluster in maize endosperm. Nucleic acids research 32: e189.
39. Sturgess R, Day P, Ellis H, Lundin K, Gjertsen, H, Kontakou, M & Ciclitira, P (1994). Wheat peptide challenge in celiac disease. Lancet 343: 756-761.
40. Tye-Din J A, Stewart J A, Dromey J A, Beissbarth T, van Heel D A, Tallham A, Henderson K, Mannering S I, Gianfranchi C, Jewel D P, Hill A U S, McCluskey J, Rossjohn J, Anderson R P (2010). Comprehensive quantitative mapping of T-cell epitopes in gluten in celiac disease. Science Translational Medicine 2 (41): 1-14.
41. Vader W, Kooy Y, Van Veelen P, et al. (2002). The gluten response in children is directed towards multiple peptides. Gastroenterol 122: 1729-1737.
42. Van de Waal, Kooy Y, Van Veelan P, et al (1999). Glutenin is involved in the gluten-driven mucosal T cell response. Eur J Immunol 29: 3133-3139.
43. Ventura A, Magazzugu G, Greco L (1999). Duration of exposure to gluten and risk for autoimmune disorders in patients with celiac disease. SIGEP Study Group for Autoimmune Disorders in Celiac Disease. Gastroenterol 117: 297-303.
44. Wieser H (1995). In: Celiac Disease (P Howdle, ed) Bailliere Clinical Gastroenterology, Vol. 9, No. 2, Bailliere Tindall, London, pp. 191-207.
45. Wieser H, Antes S, Seilmeier W (1998). Quantitative determination of gluten protein types in wheat flour by reversed-phase high-performance liquid chromatography. Cereal Chem 75: 644-65.

46. Wu, Y., D. R. Holding and J. Messing (2010). Gamma-zeins are essential for endosperm modification in quality protein maize. Proc Natl Acad Sci U S A 107: 12810-12815.
47. Wu, Y., and J. Messing (2009). Tissue-specificity of storage protein genes has evolved with younger gene copies. Maydica 54: 409-415.
48. Wu, Y., and J. Messing (2010). RNA interference-mediated change in protein body morphology and seed opacity through loss of different zein proteins. Plant Physiol 153: 337-347.
49. Wu, Y., and J. Messing (2012). RNA interference can rebalance the nitrogen sink of maize seeds without losing hard endosperm. PLoS One 7: e32850.

References for the vector shown in FIG. 7
Becker, D, Kemper, E, Schell, J, Masterson, R (1992) New plant binary vectors with selectable markers located proximal to the left T-DNA border. Plant Molecular Biology 20: 1195-1197.
Chinault, A C, Blakesley, V A, Roessler, E, Willis, D G, Smith, CA, Cook, R G, Fenwick, R G (1986) Characterization of transferable plasmids from Shigella flexneris 2a that confer resistance to trimethoprim, streptomycin, and sulfonamides. Plasmid 15: 119-131.
Condit C, Meagher R B (1983) Multiple, discrete 35S transcripts of cauliflower mosaic virus. J Mol Appl Genet 2:301-314
Depicker A, Stachel S, Dhaese P, Zambryski P, Goodman H M (1982) Nopaline synthase: transcript mapping and DNA sequence. J. Mol. Appl. Genet.1: 561-573.
Gallie, D R, Tanguay, R L, Leathers, V (1995) The tobacco etch virus 5′leader and poly (A) tail are functionally synergistic regulators of translation. Gene 165: 233-238.
Hajdukiewicz, P, Svab, Z, Maliga, P (1994) The small, versatile pPZP family of Agrobacterium binary vectors for plant transformation. Plant Molecular Biology 25: 989-994.
Itoh, Y, Haas, D (1985) Cloning vectors derived from the Pseudomonas plasmid pVS1. Gene 36: 27-36.
Jefferson, R A, Kavanagh, T A, Bevan, M W (1987) GUS fusion: β-glucuronidase as a sensitive and aversatile gene fusion marker in higher plants. EMBO J. 6: 3901-3907.
Jefferson, R A (1993) Plant promoter-alpha-glucuronidase gene construct. U.S. Pat. No. 5,268,463
Mason H S, DeWald D, Mullet J E (1993) Identificiation of a methyl jasmonate-responsive domain in the soybean vspB promoter. The Plant Cell 5: 241-251
Odell J T, Nagy F, Chua N H. (1985) Identification of DNA sequences required for activity of the cauliflower mosaic virus 35S promoter. Nature 6: 810-812
Thompson, C J, Movva, N R, Tichard, R, Crameri, R, Davies, J E, Lauwereys, M (1987) Characterization of the herbicide-resistance gene bar from Streptomyces hygroscopicus. EMBO J. 6: 2519-2523.
Vancanneyt, G, Schmidt, R, O'Connor-Sanchez, A, Willmitzer, L, Rocha-Sosa, M (1990) Construction of an intron-containing marker gene: Splicing of the intron in transgenic plants and its use in monitoring early events in Agrobacterium-mediated plant transformation. Mol Gen Genet 220: 245-250.
White, J, Chang, S Y, Bibb, M, Bibb, M (1990) A cassette containing the bar gene of S. hygroscopicus: a selectable marker for plant transformation. Nucl Acids Res 18: 1062.
Wilson, T (1999) Untranslated leader sequences from RNA viruses as enhancers of translation. U.S. Pat. No. 5,891,665
Zambryski, P, Depicker, A, Kruger, K, Goodman, H (1982) Tumor induction by Agrobacterium tumefaciens: analysis of the boundaries of T-DNA. J. Mol. Appl. Genet. 1:361-370

While the invention has been described in detail and with reference to specific examples thereof, it will be apparent to one skilled in the art that various changes and modifications can be made therein without departing from the spirit and scope thereof

In the claims:

1. A method for detecting CD-inducing epitopes in wheat glutenin and gliadin proteins comprising:
a) contacting gluten sensitive T cells with glutenin and gliadin synthetic peptides obtained from deep sequencing wheat cultivars
b) determining the SI index and/or enterocyte cell height (ECH), of said cells in the presence or absence of said fragments, fragments which reduce ECH or stimulate sensitive T cell proliferation being associated with the occurrence of CD.

2. The method of claim 1, wherein step a) comprises contacting said cells with a pooled population of said synthetic peptides.

3. The method of claim 2, further comprising altering the nucleic acids encoding said CD-inducing epitopes of said wheat glutenin and gliadin proteins, such that they no longer reduce ECH or stimulate sensitive T cell proliferation.

4. A plurality of recombinant wheat and gliadin encoding nucleic acids produced by the method of claim 3 for expression of glutenins and gliadin proteins which lack CD-inducing epitopes.

5. A method for the production of transgenic maize, which expresses wheat glutenin and gliadin proteins lacking CD inducing epitopes comprising:
a) introducing DNA constructs comprising sequences encoding one or more wheat glutenin or gliadin proteins, said sequences being altered such that the encoded proteins lack native CD-inducing epitopes, said construct optionally comprising a selectable marker suitable for isolation of transgenic cells,
b) propagating said isolated cells to generate a transgenic maize plant; and
c) obtaining flour from said plants for use in baking consumable products, said products lacking CD inducing epitopes and thereby being safe to consume by patients exhibiting gluten intolerance.

6. The method of claim 5, further comprising back crossing the first transgenic plant of step c) with a separate second transgenic plant expressing at least one different recombinant glutenin or gliadin protein, thereby producing a plant expressing altered glutenins and gliadins from said first and second plants.

7. The method of claim 5 further comprising introducing at least one RNAi construct into said plant, said RNAi molecule being effective to down modulate production of at least one zein protein.

8. The method of claim 5, wherein said transgenic maize is obtained from a high quality protein maize line.

9. Flour obtained from the transgenic maize of the plant of claim 5, wherein said flour comprises at least one recombinant glutenin.

10. A plant or progeny thereof obtained from the method of claim 5, wherein said plant or progeny comprises at least one recombinant glutenin.

11. Flour obtained from the transgenic maize of the plant of claim 6, wherein said flour comprises at least one recombinant glutenin.

12. Flour obtained from the transgenic maize of the plant of claim 7, wherein said flour comprises at least one recombinant glutenin.

13. Flour obtained from the transgenic maize of the plant of claim 8, wherein said flour comprises at least one recombinant glutenin.

14. A plant or progeny thereof obtained from the method of claim 6, wherein said plant or progeny comprises at least one recombinant glutenin.

15. A plant or progeny thereof obtained from the method of claim 7, wherein said plant or progeny comprises at least one recombinant glutenin.

16. A plant or progeny thereof obtained from the method of claim 8, wherein said plant or progeny comprises at least one recombinant glutenin.
